SEQUENCE LISTING (8 sequences)

SEQ ID NO: 1 - DNA, 2598 bp, Artificial Sequence
  TFL
atgctactagtaaatcagtcacaccaaggcttcaataaggaacacacaagcaagatggtatacgatgatcatttagtcagtgtggttccgaagttattccttgtgtgttcgttctaccatagcgctattgttttatatgtgcttttggcggcggcggcgcattctgcctttgcggcggattcgcgataacaaaatatacacgaaaaccgccgccgccgcgtaagacggaaacgccgcctacccgagccctgcgtggaggtggttcctaatattacttatcaatgcatggagctgaatttcgggctcgggacgcacctccaccaaggattataatgaatagttacgtacctcgacttaaagtacaaaatccccgacaacctccccttctcaaccaagaacctggacctgagctttaatcccatgttttaggggctgttggaggggaagagttggttcttggacctggactcgaaattagggctgaggcatttaggcagctatagcttcttcagtttcccagaactgcaggtgctggatttagactccgtaaatccgtcgatatcgaagaagtcaaagggtcttgacgtccacgacctaaattccaggtgtgaaatccagacaattgaagatggggcatatcagagcctaagccacctctctaggtccacactttaggtctgttaacttctaccccgtatagtctcggattcggtggagagaaccttaatattgacaggaaaccccatccagagtttagccctgggagccttttctggactatggaattataactgtcctttggggtaggtctcaaatcgggaccctcggaaaagacctgattcaagtttacagaagctggtggctgtggagacaaatctagcatctctagagaacttccccagttcaaatgtcttcgaccaccgacacctctgtttagatcgtagagatctcttgaaggggattggacatctcaaaactttgaaagaacttaatgtggctcacaatcttatccaatctttctaacctgtagagttttgaaactttcttgaattacaccgagtgttagaataggttagaaagaaattacctgagtatttttctaatctgaccaatctagagcacttggacctttccagcaactttaatggactcataaaaagattagactggttagatctcgtgaacctggaaaggtcgttgaagattcaaagtatttattgcacagacttgcgggttctacatcaaatgcccctactcaatttctaagtttcataaataacgtgtctgaacgcccaagatgtagtttacggggatgagttactctctttagacctgtccctgaaccctatgaactttatccaaccaggtgcatttaaagaagagagaaatctggacagggacttgggatacttgaaataggttggtccacgtaaatttcttattaggcttcataagctgactttaagaaataattttgatagtttaaatgtaatgaaaacttaatccgaagtattcgactgaaattctttattaaaactatcaaatttacattacttttgatgtattcaaggtctggctggtttagaagtccatcgtttggttctgggagaatttagaaatacataagttccagaccgaccaaatcttcaggtagcaaaccaagaccctcttaaatctttagaaggaaacttggaaaagtttgacaaatctgctctagagggcctgtgcaatttgaccattcttcctttgaaccttttcaaactgtttagacgagatctcccggacacgttaaactggtaagaagaattccgattagcatacttagactactacctcgatgatattattgacttatttaatcttcttaaggctaatcgtatgaatctgatgatggagctactataataactgaataaattatgtttgacaaatgtttcttcattttccctggtgagtgtgactattgaaagggtaaaagacacaaactgtttacaaagaagtaaaagggaccactcacactgataactttcccattttctgttttcttataatttcggatggcaacatttagaattagttaactgtaaatttggacagtttaaaagaatattaaagcctaccgttgtaaatcttaatcaattgacatttaaacctgtcaaacccacattgaaactcaaatctctcaaaaggcttactttcacttccaacaaaggtgggaatgggtgtaactttgagtttagagagttttccgaatgaaagtgaaggttgtttccacccttagctttttcagaagttgatctaccaagccttgagtttctagatctcagtagaaatggcttgcgaaaaagtcttcaactagatggttcggaactcaaagatctagagtcatctttaccgaacagtttcaaaggttgctgttctcaaagtgattttgggacaaccagcctaaagtatttagattcaaagtttccaacgacaagagtttcactaaaaccctgttggtcggatttcataaatctactgagcttcaatggtgttattaccatgagttcaaacttcttgggcttagaacaactagaagactcgaagttaccacaa

SEQ ID NO: 2 - protein, 865 aa, Artificial Sequence
  TLF
MLLVNQSHQGFNKEHTSKMVSAIVLYVLLAAAAHSAFAADPEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLKELALDTNQLKSVPDGIFDRLTSLQKIWLHTNPWDCSCPRIDYLSRWLNKNSQKEQGSAKCSGSGKPVRSIICPTLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1644 bp, Artificial Sequence
  TOY3
atgctactagtaaatcagtcacaccaaggcttcaataaggaacacacaagcaagatggtatacgatgatcatttagtcagtgtggttccgaagttattccttgtgtgttcgttctaccatagcgctattgttttatatgtgcttttggcggcggcggcgcattctgcctttgcggcggattcgcgataacaaaatatacacgaaaaccgccgccgccgcgtaagacggaaacgccgcctacccgagccctgcgtggaggtggttcctaatattacttatcaatgcatggagctgaatttcgggctcgggacgcacctccaccaaggattataatgaatagttacgtacctcgacttaaagtacaaaatccccgacaacctccccttctcaaccaagaacctggacctgagctttaatcccatgttttaggggctgttggaggggaagagttggttcttggacctggactcgaaattagggctgaggcatttaggcagctatagcttcttcagtttcccagaactgcaggtgctggatttagactccgtaaatccgtcgatatcgaagaagtcaaagggtcttgacgtccacgacctaaattccaggtgtgaaatccagacaattgaagatggggcatatcagagcctaagccacctctctaggtccacactttaggtctgttaacttctaccccgtatagtctcggattcggtggagagaaccttaatattgacaggaaaccccatccagagtttagccctgggagccttttctggactatggaattataactgtcctttggggtaggtctcaaatcgggaccctcggaaaagacctgattcaagtttacagaagctggtggctgtggagacaaatctagcatctctagagaacttccccagttcaaatgtcttcgaccaccgacacctctgtttagatcgtagagatctcttgaaggggattggacatctcaaaactttgaaagaacttaatgtggctcacaatcttatccaatctttctaacctgtagagttttgaaactttcttgaattacaccgagtgttagaataggttagaaagaaattacctgagtatttttctaatctgaccaatctagagcacttggacctttccagcaactttaatggactcataaaaagattagactggttagatctcgtgaacctggaaaggtcgttgaagattcaaagtatttattgcacagacttgcgggttctacatcaaatgcccctactcaatttctaagtttcataaataacgtgtctgaacgcccaagatgtagtttacggggatgagttactctctttagacctgtccctgaaccctatgaactttatccaaccaggtgcatttaaagaagagagaaatctggacagggacttgggatacttgaaataggttggtccacgtaaatttcttattaggctcaaagagctcgccctggacaccaaccagctgaagtctgttcctgatgggatttaatccgagtttctcgagcgggacctgtggttggtcgacttcagacaaggactaccctaatttgatcgcctgaccagcttgcagaaaatttggcttcatacaaacccttgggactgcagtaaactagcggactggtcgaacgtc

SEQ ID NO: 4 - protein, 547 aa, Artificial Sequence
  TOY3
MLLVNQSHQGFNKEHTSKMVSAIVLYVLLAAAAHSAFAADPEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLKELALDTNQLKSVPDGIFDRLTSLQKIWLHTNPWDCSCPRIDYLSRWLNKNSQKEQGSAKCSGSGKPVRSIICPTLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 2526 bp, Artificial Sequence
  TOY8
atgctactagtaaatcagtcacaccaaggcttcaataaggaacacacaagcaagatggtatacgatgatcatttagtcagtgtggttccgaagttattccttgtgtgttcgttctaccatagcgctattgttttatatgtgcttttggcggcggcggcgcattctgcctttgcggcggattcgcgataacaaaatatacacgaaaaccgccgccgccgcgtaagacggaaacgccgcctacccgagccctgcgtggaggtggttcctaatattacttatcaatgcatggagctgaatttcgggctcgggacgcacctccaccaaggattataatgaatagttacgtacctcgacttaaagtacaaaatccccgacaacctccccttctcaaccaagaacctggacctgagctttaatcccatgttttaggggctgttggaggggaagagttggttcttggacctggactcgaaattagggctgaggcatttaggcagctatagcttcttcagtttcccagaactgcaggtgctggatttagactccgtaaatccgtcgatatcgaagaagtcaaagggtcttgacgtccacgacctaaattccaggtgtgaaatccagacaattgaagatggggcatatcagagcctaagccacctctctaggtccacactttaggtctgttaacttctaccccgtatagtctcggattcggtggagagaaccttaatattgacaggaaaccccatccagagtttagccctgggagccttttctggactatggaattataactgtcctttggggtaggtctcaaatcgggaccctcggaaaagacctgattcaagtttacagaagctggtggctgtggagacaaatctagcatctctagagaacttccccagttcaaatgtcttcgaccaccgacacctctgtttagatcgtagagatctcttgaaggggattggacatctcaaaactttgaaagaacttaatgtggctcacaatcttatccaatctttctaacctgtagagttttgaaactttcttgaattacaccgagtgttagaataggttagaaagaaattacctgagtatttttctaatctgaccaatctagagcacttggacctttccagcaactttaatggactcataaaaagattagactggttagatctcgtgaacctggaaaggtcgttgaagattcaaagtatttattgcacagacttgcgggttctacatcaaatgcccctactcaatttctaagtttcataaataacgtgtctgaacgcccaagatgtagtttacggggatgagttactctctttagacctgtccctgaaccctatgaactttatccaaccaggtgcatttaaagaagagagaaatctggacagggacttgggatacttgaaataggttggtccacgtaaatttcttattaggcttcataagctgactttaagaaataattttgatagtttaaatgtaatgaaaacttaatccgaagtattcgactgaaattctttattaaaactatcaaatttacattacttttgatgtattcaaggtctggctggtttagaagtccatcgtttggttctgggagaatttagaaatacataagttccagaccgaccaaatcttcaggtagcaaaccaagaccctcttaaatctttagaaggaaacttggaaaagtttgacaaatctgctctagagggcctgtgcaatttgaccattcttcctttgaaccttttcaaactgtttagacgagatctcccggacacgttaaactggtaagaagaattccgattagcatacttagactactacctcgatgatattattgacttatttaatcttcttaaggctaatcgtatgaatctgatgatggagctactataataactgaataaattatgtttgacaaatgtttcttcattttccctggtgagtgtgactattgaaagggtaaaagacacaaactgtttacaaagaagtaaaagggaccactcacactgataactttcccattttctgttttcttataatttcggatggcaacatttagaattagttaactgtaaatttggacagtttaaaagaatattaaagcctaccgttgtaaatcttaatcaattgacatttaaacctgtcaaacccacattgaaactcaaatctctcaaaaggcttactttcacttccaacaaaggtgggaatgggtgtaactttgagtttagagagttttccgaatgaaagtgaaggttgtttccacccttagctttttcagaagttgatctaccaagccttgagtttctagatctcagtagaaatggcttgcgaaaaagtcttcaactagatggttcggaactcaaagatctagagtcatctttaccgaacagtttcaaaggttgctgttctcaaagtgattttgggacaaccagcctaaagtatttagattcaaagtttccaacgacaagagtttcactaaaaccctgttggtcggatttcataaatctactgagc

SEQ ID NO: 6 - protein, 841 aa, Artificial Sequence
  TOY8
MLLVNQSHQGFNKEHTSKMVSAIVLYVLLAAAAHSAFAADPEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMASNQLKSVPDGIFDRLTSLQKIWLHTNPWDCSCPRIDYLSRWLNKNSQKEQGSAKCSGSGKPVRSIICPTLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 2598 bp, Artificial Sequence; identical to SEQ ID NO: 1
  TOY9

SEQ ID NO: 8 - protein, 865 aa, Artificial Sequence; identical to SEQ ID NO: 2
  TOY9

(19) United States
(12) Patent Application Publication
(10) Pub. No.: US 2009/0312249 A1
(43) Pub. Date: Dec. 17, 2009

(54) TLR4 decoy receptor protein

(75) Inventors: Keehoon Jung, Seoul (KR); HoMin Kim, Kyungbuk (KR); Hak-Zoo Kim, Seoul (KR); Jung-Eun Lee, Daejeon (KR); Gyun Min Lee, Daejeon (KR); Sun Chang Kim, Daejeon (KR); Jie-Oh Lee, Daejeon (KR); Hak-Sung Kim, Daejeon (KR); Gou Young Koh, Daejeon (KR)

Correspondence Address: JHK LAW, P.O. BOX 1078, LA CANADA, CA, 91012-1078, US

(73) Assignee: Korea Advanced Institute of Science and Technology

(21) Appl. No.: 12/315,989

(22) Filed: Dec. 8, 2008

Related U.S. Application Data
(60) Provisional application No. 61/012,024, filed on Dec. 6, 2007.

(51) Int. Cl. A61K 38/17; C12N 15/12; C12N 15/85; C12N 15/74; C12N 1/19; C12N 1/21; C12N 5/10; C12P 21/04; C07K 14/47; A61P 29/00; A61P 31/04

(52) U.S. Cl. 514/12; 536/23.4; 435/320.1; 435/252.3; 435/254.2; 435/348; 435/325; 435/69.7; 530/350

(57) ABSTRACT

The present application discloses a nucleic acid molecule encoding a fusion polypeptide capable of binding myeloid differentiation protein-2 (MD-2) polypeptide, which includes: a nucleotide sequence encoding an MD-2 polypeptide-binding portion of human toll-like receptor 4 (TLR4), a leucine-rich repeats (LRR) module, and a multimerizing component.

### BACKGROUND OF THE INVENTION

1. Field of the Invention

The present invention relates to fusion proteins capable of binding MD-2, namely “Toll-like receptor 4 (TLR4) decoy receptor protein (TOY)” based on an application of “Hybrid LRR technique”. TOY is disclosed which are therapeutically useful for treating disorders associated with TLR4 signaling such as sepsis, septic shock caused by Gram-negative infections accompanying a profound vasodilation, extravascular plasma leakage resulting from an increase in endothelial permeability, sterile inflammation, and rheumatoid arthritis.

2. Description of the Background

Lipopolysaccharide (LPS) derived from the cell wall of the Gram-negative bacteria is a main component to trigger host innate immune responses followed by sepsis or septic shock. Several host humoral and cell-surface proteins participate in the innate recognition of LPS, including the LPS-binding protein (LBP), the CD14, the TLR4, and the myeloid differentiation protein-2 (MD-2) (Pugin J, et al., Blood, 2004; 104:4071-4079). TLR4 is a type I transmembrane glycoprotein characterized by the presence of 22 leucine-rich repeats (LRRs) on the extracellular domain with horseshoe-like shapes and the cytoplasmic Toll-IL-1 resistance (TIR) domain that can dimerize to initiate the signaling (Visintin A, et al., The Journal of Immunology, 2005; 175:6465-6472). In myeloid cells, the LPS receptor complex is composed of CD14, TLR4, and MD-2, whereas endothelial cells express TLR4 and MD-2, and epithelial cells only express TLR4 (Aderem A, et al., Nature, 2000; 406:782-787). Response of endothelial cells to LPS requires the presentation of LPS to the TLR4/MD-2 membrane receptor complex by LBP and soluble CD14 (Henneke P, et al., Critical Care Medicine, 2002; 30:S207-S213).

MD-2 is an Ig domain-folded glycoprotein belonging to the ML (MD-2-related lipid recognition) family of lipid binding receptors which binds to LPS and confers LPS responsiveness to TLR4-expressing cells. The crystal structure of TLR4/MD-2/Eritoran complex shows that MD-2 has a barrel-like structure with a hydrophobic cavity large enough to accommodate the fatty acid moieties of lipid A (Kim H M, et al., Cell, 2007; 130:906-917). Only monomeric MD-2 which interacts with LPS or TLR4 is the functional form (Visintin A, et al., The Journal of Immunology, 2005; 175:6465-6472). Some of the MD-2 molecules remain attached to TLR4, whereas the rest are secreted in the extracellular milieu.

Because whole and truncated forms of TLR4 have their intrinsic biochemical properties, their expression and production in any cell type is low, they are insoluble and purification yields of those proteins are extremely low. These problems have been major obstacles to obtaining mass production of soluble and functional forms of TLR4 protein or truncated TLR4 protein in their biological and medical applications.

Recently, a technique called the “Hybrid LRR technique” (described in PCT/KR2008/5220, incorporated by reference herein in its entirety, particularly with reference to hybrid LRR technique) has been developed to overcome these problems. Variable lymphocyte receptors (VLRs) are a new type of immune receptors in jawless fish, which resemble the adaptive immune receptors in jawed vertebrates. VLRs and TLRs commonly contain the LRR domain in the extracellular fragment, which is composed of a signal sequence, an N-terminal cap (LRRNT), several LRR modules, and a C-terminal cap (LRRCT). Therefore, stable TLR4-VLR hybrid proteins can be generated without any loss of the intrinsic structural integrity of TLR4 by replacing some LRR modules and LRRCT of TLR4 with those of VLR. A series of hybrids of human TLR4 and hagfish VLR-B.61 called the “TV hybrids” were generated. The TV3 consists of hTLR4 amino acids from 27 to 227 and VLR-B.61 amino acids from 125 to 200, the TV8 consists of hTLR4 amino acids from 27 to 527 and VLRB.61 amino acids from 133 to 200, and the TV9 consists of hTLR4 amino acids from 27 to 545 and VLB.61 amino acids from 125 to 200, respectively (Kim H M, et al., Cell, 2007; 130:906-917).

The N-terminal region of the TLR4 ectodomain is minimally required for MD-2 binding shown by crystal structure analysis. The purpose for development of TOY is that as a decoy receptor of TLR4, it binds to the soluble MD-2 interacting with LPS. When the bacterial infection is rapidly progressed, all the TOY proteins which contain a minimal domain for MD-2 binding can interact with MD-2 or MD-2/LPS complex instead of the intrinsic native TLR4. Therefore, TOY could inhibit TLR4 activation and the subsequent sepsis or septic shock.

Activity of TOY proteins can be assessed in in vivo by administration of TOY before and after injection of LPS into the animal or by administration of TOY into the cecal ligation and puncture peritonitis mouse model.

### SUMMARY OF THE INVENTION

The present invention provides for nucleic acid molecules and multimeric fusion proteins capable of binding MD-2, namely “TLR4 decoy receptor proteins (TOY)”. TOY are disclosed which are therapeutically useful for treating disorders associated with TLR4 signaling such as sepsis, septic shock caused by Gram-negative bacterial infections accompanying a profound vasodilation and extravascular plasma leakage resulting from an increase in endothelial permeability, as well as sterile inflammation and rheumatoid arthritis.

In one aspect, the present invention is directed to an isolated nucleic acid molecule encoding a polypeptide capable of binding MD-2 polypeptide, which includes a nucleotide sequence encoding a TLR4 component and VLR-B.61 component. A nucleotide sequence encoding a multimerizing component may be linked to a nucleotide sequence encoding a TLR4 component and VLR-B.61 component. And the multimerizing component may be an immunoglobulin domain. In one aspect, the immunoglobulin domain may be the Fc domain of IgG, the heavy chain of IgG, or the light chain of IgG.

In another aspect, in the nucleic acid molecule, the TLR4 component may include a nucleotide sequence encoding the amino acid sequences of ectodomain of TLR4.

In another aspect, the invention is directed to an isolated nucleic acid molecule comprising a nucleotide sequence encoding:

(a) the nucleotide sequence set forth in Table 1 referred to as TFL (Fc-tagged hTLR4 full-length ectodomain), which includes hTLR4 amino acids from 27 to 631 (nucleotides from 79 to 1893) and Fc domain of human IgG;

(b) the nucleotide sequence set forth in Table 1 referred to as TOY3, which includes hTLR4 amino acids from 27 to 227 (nucleotides from 79 to 681), VLRB.61 amino acids from 125 to 200 (nucleotides 373 to 600), and Fc domain of human IgG;

(c) the nucleotide sequence set forth in Table 1 referred to as TOY8, which includes hTLR4 amino acids from 27 to 527 (nucleotides from 79 to 1581), VLRB.61 amino acids from 133 to 200 (nucleotides from 397 to 600), and Fc domain of human IgG;

(d) the nucleotide sequence set forth in Table 1 referred to as TOY9, which includes hTLR4 amino acids from 27 to 545 (nucleotides from 79 to 1635), VLRB.61 amino acids from 125 to 200 (nucleotides from 373 to 600), and Fc domain of human IgG; or

(e) a nucleotide sequence which, as a result of the degeneracy of the genetic code, differs from the nucleotide sequence of (a), (b), (c), or (d) but which encodes identical amino acid sequence as expressed therefrom.

The invention is also directed to a vector that includes all of the nucleic acid molecules described above. The vector may be an expression vector.

The invention is also directed to a host-vector system for the production of a fusion polypeptide which includes the expression vector described above in a suitable host cell. Such a suitable host cell may include a bacterial cell, yeast cell, insect cell, or mammalian cell.

The invention is also directed to a fusion polypeptide encoded by any of the isolated nucleic acid molecules described above, including, but not limited to the amino acid sequence for TFL, TOY3, TOY8 and TOY9.

The invention is also directed to a composition capable of binding MD-2 molecule to form a nonfunctional complex comprising a multimer of the fusion polypeptide described above including, but not limited to, those fusion constructs that use TLR4 components. In particular, the multimer may be a dimer.

In another aspect, the invention is directed to a method of producing a fusion polypeptide which includes growing cells of the host-vector system described above, under conditions permitting production of the fusion polypeptide and recovering the fusion polypeptide so produced. Such a fusion polypeptide may be modified by acetylation or pegylation. The acetylation may be accomplished with a molar excess of acetylation reagent ranging from at least about a 10 fold molar excess to about a 100 fold molar excess. The pegylation may be with 10K or 20K PEG.

In still another aspect, the invention is directed to a method of inhibiting developing sepsis, which includes administering to a mammal in need thereof an effective amount of the fusion polypeptide described herein. In a preferred embodiment, the developing sepsis may be due to penetrating trauma to the abdomen, heart valve disease, a large bowel incarceration and other biomedical complications.

In still another aspect, the invention is directed to a method of inhibiting sepsis and dampening signaling pathways for sepsis following symptoms, which includes administering to a mammal in need thereof an effective amount of the fusion polypeptide described herein. In a preferred embodiment, symptoms of sepsis may include fever, chills, shaking, weakness, nausea, vomiting, confusion, and diarrhea.

In still another aspect, the invention is directed to a method of inhibiting septic shock and dampening signaling pathways following symptoms, which includes administering to a mammal in need thereof an effective amount of the fusion polypeptide described herein. In a preferred embodiment, symptoms of septic shock may include confusion, decreased consciousness, shaking chills, abnormal body temperature, flushed skin, pounding pulse, rapid breathing, blood pressure that rise and falls and/or extremities that are cool, pale, and bluish.

In still another aspect, the invention is directed to a method of decreasing or inhibiting plasma leakage and/or formation of thrombosis in a mammal, which includes administering to a mammal in need thereof an effective amount of the fusion polypeptide described herein. In a preferred embodiment, the leakage and/or formation of thrombosis may be in the multiple organs.

In still another aspect, the invention is directed to a method of decreasing several proinflammatory cytokines levels in a mammal, which includes administering to a mammal in need thereof an effective amount of the fusion polypeptide described herein. In a preferred embodiment, the cytokines may be in the plasma and/or serum.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1A shows a schematic diagram of how MD2 in blood binds to TLR4, which is located on the cell surface. MD-2-free form of TLR4, MD-2-bound form are shown. Circulating MD-2 in blood can interact with TLR4. In myeloid cells, the LPS receptor complex is composed of CD14, TLR4, and MD-2, whereas endothelial cells express TLR4 and MD-2, and epithelial cells express only TLR4.

FIG. 1B shows structural interactions for binding between LPS and MD-2 and between MD-2 and TOY3 or TOY8. The N-terminal portion of the ectodomain of TLR4 is significant for MD-2 binding.

FIG. 2 shows schematic diagram of molecular constructs and structures for each indicated TOY protein. Variable sizes of TLR4 fragments, VLR-B.61 fragments, and human IgG Fc (hIgG-Fc) are shown. hIgG-Fc is fused with the C-terminal region of hTLR4/VLR-B.61 hybrid. Here, TFL (Fc-tagged hTLR4 full-length ectodomain) includes hTLR4 amino acids from 27 to 631 (nucleotides from 79 to 1893) and Fc domain of human IgG; TOY3 includes hTLR4 amino acids from 27 to 227 (nucleotides from 79 to 681), VLRB.61 amino acids from 125 to 200 (nucleotides 373 to 600), and Fc domain of human IgG; TOY8 includes hTLR4 amino acids from 27 to 527 (nucleotides from 79 to 1581), VLRB.61 amino acids from 133 to 200 (nucleotides from 397 to 600), and Fc domain of human IgG; TOY9 includes hTLR4 amino acids from 27 to 545 (nucleotides from 79 to 1635), VLRB.61 amino acids from 125 to 200 (nucleotides from 373 to 600), and Fc domain of human IgG.

FIG. 3A shows Western blot analysis for the detection of expression pattern of TFL and each exemplified TOY protein. TFL and each exemplified TOY protein from the supernatants of transiently transfected HEK 293 cells were immunoprecipitated with a protein A bead. Each sample was mixed with sample buffer, heat-denatured for 10 min, run on 10% SDS-PAGE, and electro-blotted onto nitrocellulose membranes. The membrane was probed with HRP-conjugated anti-Fc antibody to detect Fc-fused proteins.

FIG. 3B shows size and dimeric status of TFL, TOY3, and TOY8. These proteins were produced in CHO cells, purified with Protein-A sepharose affinity chromatography, ˜2 μg loaded under reducing (R) and nonreducing (NR) condition into SDS-PAGE, and stained with Coomassie blue. Estimated molecular size of each recombinant protein under reducing and nonreducing condition were ˜110 kDa, ˜220 kDa for TFL, ˜105 kDa, ˜210 kDa for TOY8, and ˜65 kDa, ˜130 kDa for TOY3, respectively.

FIG. 4A shows SDS-PAGE analysis of the TLR4 hybrid/MD-2 complexes. Protein A-tagged MD-2 and the TV3, TV8, TV9, or hTLR4 ectodomain were co-infected into insect cells, subsequently purified using IgG Sepharose (GE Healthcare) affinity chromatography, and analyzed under SDS-PAGE. Each of the TV hybrids or hTLR4 ectodomain and MD-2 proteins formed a stable 1:1 complex and were not separated during purification. Therefore, all recombinant TLR4/VLR-B.61 fusion hybrid proteins were capable of binding MD-2.

FIG. 4B shows virtual isoelectric point value of TOY3 produced in CHO cells. Virtual isoelectric point value of TOY3 produced in CHO cells was analyzed with IsoGel Agarose IEF plate (Cambrex, N.J., USA). TOY3 resulted in pI value of about 5.2, which is much lower than its theoretical value, which is mainly due to abundant glycosylation process in CHO cells.

FIG. 5 shows BIAcore analysis of the interaction between MD-2 and each exemplified TOY protein. Analyses for those interactions revealed that K_{D} of TFL to MD-2 was ˜81 nM and that of TOY3 to MD-2 was ˜76 nM, whereas K_{D} of TOY8 to MD-2 was ˜56 nM. Therefore, fusion of the VLR-B.61 component to recombinant TLR4 protein did not alter the binding affinity of the intrinsic native TLR4 protein not bearing VLR-B.61 portion.

FIG. 6A shows survival curves demonstrating effects of TOY3 and Fc (control protein) on LPS-induced sepsis in a mouse model for lethality experiment. TOY3 (20 mg/kg) and Fc (20 mg/kg) was pretreated into intraperitoneal cavity of C³H/HeN mice, and then 10 minutes later, LPS (15 mg/kg) was administered intraperitoneally into the mice. TOY3-treated mice showed prolonged lifespan compared to Fc-treated mice (P<0.001), therefore, TOY3 has a prominent preventive effect in LPS-induced sepsis in a mouse model.

FIG. 6B shows survival curves demonstrating effects of TOY3 and Fc (control) on LPS-induced sepsis in a mouse model for lethality experiment. LPS (15 mg/kg) was administered into intraperitoneal cavity of C³H/HeN mice, and then 1 hour later, TOY3 (20 mg/kg) and Fc (20 mg/kg) was posttreated intraperitoneally into the mice. TOY3 also showed prominent therapeutic effect in LPS-induced sepsis in a mouse model (TOY3 versus Fc, P<0.001). Also, this experiment implies that the A patch of the TLR4 ectodomain is sufficient for the MD-2 binding in vivo, because TOY3 bearing only the A patch showed significant effect on treatment of sepsis.

FIG. 6C shows survival curves demonstrating effects of TOY3 and Fc (control) on cecal ligation and puncture (CLP)-induced sepsis in a mouse model for lethality experiment. TOY3 (20 mg/kg) and Fc (20 mg/kg) was pretreated into intraperitoneal cavity of C³H/HeN mice, and then 1 hour later, CLP procedure was performed. This result shows that TOY3-treated mice showed prolonged lifespan compared to Fc-treated mice (P<0.005), therefore, TOY3 also has preventive effect even in CLP-induced sepsis in a mouse model.

FIG. 6D shows survival curves demonstrating effects of TOY3 and Fc (control) on CLP-induced sepsis in a mouse model for lethality experiment. CLP procedure was performed in C³H/HeN mice, and then 1 hour later, TOY3 (20 mg/kg) and Fc (20 mg/kg) was posttreated intraperitoneally into the mice. TOY3 has not only preventive but also promising therapeutic effect in CLP-induced sepsis in a mouse model compared to control Fc (P<0.005).

FIG. 7 shows effects of TOY3 and Fc (control) on organ damage in LPS-induced sepsis in a mouse model. Because not only pretreatment but also posttreatment of TOY3 showed significant improving effect in the lethality experiments, LPS (8 mg/kg) was administered into intraperitoneal cavity of C³H/HeN mice, and then 1 hour later, TOY3 (20 mg/kg) and Fc (20 mg/kg) was posttreated intraperitoneally into the mice. Livers and lungs were harvested from mice and subsequent fixation, paraffin section and H&E staining were performed. The number of thrombi in liver of TOY3-treated mice was significantly lower than that of Fc-treated mice. There were few RBCs accumulated in many vessels in TOY3-treated mice similar to normal control mice. In lung of Fc-treated mice, a large number of intravascular thrombi were observed around alveoli structures. Presence of intravascular thrombi in lung of TOY3-treated mice was hardly detected compared to that of Fc-treated mice. TOY3 also has therapeutic effect on improving organs damage in LPS-induced sepsis in a mouse model.

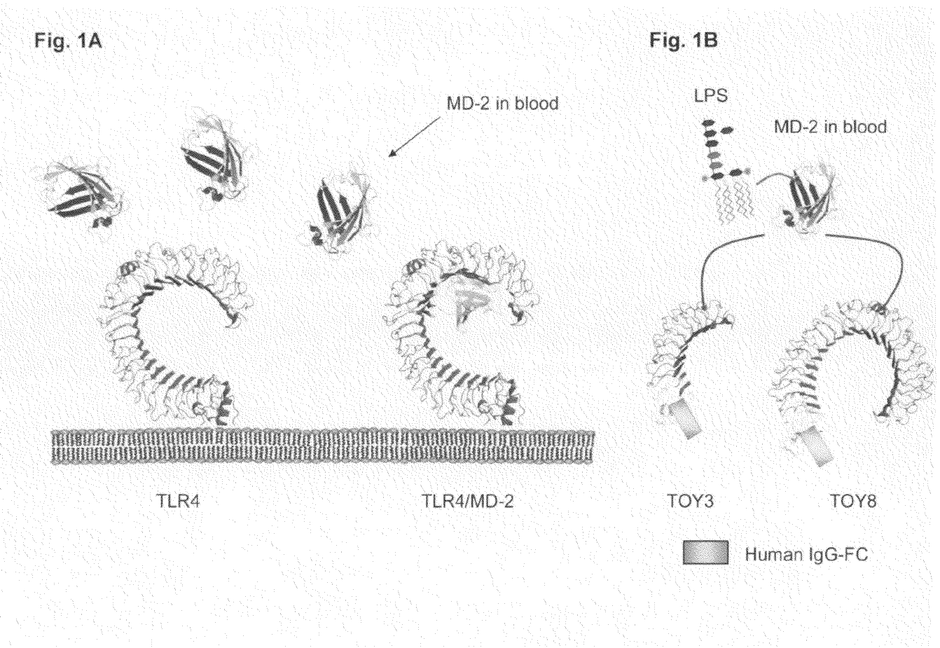

TABLE 1 shows the nucleic acid and amino acid sequences of TFL, TOY3, TOY8, and TOY9. In particular, SEQ ID NO:1 represents the sense strand of the nucleic acid depicted for TFL. SEQ ID NO:2 represents the amino acid sequence depicted for TFL.

Nucleic acid residue no. 115 to 1908 of SEQ ID NO:1 (corresponding to amino acid nos. 39 to 636 of SEQ ID NO:2) encodes the TLR4 portion; and nucleic acid residue no. 1909 to 2598 of SEQ ID NO:1 (corresponding to amino acid nos. 637 to 866 of SEQ ID NO:2) encodes the hIgG-Fc portion.

SEQ ID NO:3 represents the sense strand of the nucleic acid depicted for TOY3. SEQ ID NO:4 represents the amino acid sequence depicted for TOY3. Nucleic acid residue no. 115 to 726 of SEQ ID NO:3 (corresponding to amino acid nos. 39 to 242 of SEQ ID NO:4) encodes the TLR4 portion; nucleic acid residue no. 727 to 954 of SEQ ID NO:3 (corresponding to amino acid nos. 243 to 318 of SEQ ID NO:4) encodes the VRLB.61 portion; and nucleic acid residue no. 955 to 1641 of SEQ ID NO:3 (corresponding to amino acid nos. 319 to 547 of SEQ ID NO:4) encodes the hIgG-Fc portion.

SEQ ID NO:5 represents the sense strand of the nucleic acid depicted for TOY8. SEQ ID NO:6 represents the amino acid sequence depicted for TOY8. Nucleic acid residue no. 115 to 1623 of SEQ ID NO:5 (corresponding to amino acid nos. 39 to 541 of SEQ ID NO:6) encodes the TLR4 portion; nucleic acid residue no. 1624 to 1836 of SEQ ID NO:5 (corresponding to amino acid nos. 542 to 612 of SEQ ID NO:6) encodes the VRLB.61 portion; and nucleic acid residue no. 1837 to 2523 of SEQ ID NO:5 (corresponding to amino acid nos. 613 to 841 of SEQ ID NO:6) encodes the hIgG-Fc portion.

SEQ ID NO:7 represents the sense strand of the nucleic acid depicted for TOY9. SEQ ID NO:8 represents the amino acid sequence depicted for TOY9. Nucleic acid residue no. 115 to 1680 of SEQ ID NO:7 (corresponding to amino acid nos. 39 to 560 of SEQ ID NO:8) encodes the TLR4 portion; nucleic acid residue no. 1681 to 1908 of SEQ ID NO:7 (corresponding to amino acid nos. 561 to 636 of SEQ ID NO:8) encodes the VRLB.61 portion; and nucleic acid residue no. 1909 to 2595 of SEQ ID NO:7 (corresponding to amino acid nos. 637 to 865 of SEQ ID NO:8) encodes the hIgG-Fc portion.

### DETAILED DESCRIPTION OF THE INVENTION

In the present application, “a” and “an” are used to refer to both single and a plurality of objects.

As used herein, “about” or “substantially” generally provides a leeway from being limited to an exact number. For example, as used in the context of the length of a polypeptide sequence, “about” or “substantially” indicates that the polypeptide is not to be limited to the recited number of amino acids. A few amino acids add to or subtracted from the N-terminus or C-terminus may be included so long as the functional activity such as its binding activity is present.

As used herein, “A patch” or “B patch” refers to the surface of TLR4 that interacts with MD-2, which has a long and narrow shape with dimensions 40×20 Å as discussed in Kim H M, et al., Cell, 2007; 130:906-917, incorporated by reference in its entirety, but especially with respect to the description of the structure of TLR4 and MD-2 binding and description of the “patches”. It can be divided into two chemically and evolutionarily distinct areas, the A and B patches. The A patch is negatively charged and evolutionarily conserved, whereas the B patch is positively charged and located in a less conserved area, although the residues directly interacting with MD-2 are strictly conserved. The A and B patches of TLR4 are composed of the residues in the concave surface derived from the “LxLxxN” part of the LRR modules in the N-terminal domain and of the central domain, respectively. The interaction between TLR4 and MD-2 is mediated by an extensive network of charge-enhanced hydrogen bonds. The negatively charged residues in the A patch interact with the positively charged Arg68 and Lys 109 residues in MD-2. The positively charged B patch interacts with negatively charged residues in the loop between the βF strand and the α helix of MD-2.

As used herein, administration “in combination with” one or more further therapeutic agents includes simultaneous (concurrent) and consecutive administration in any order.

As used herein, “amino acid” and “amino acids” refer to all naturally occurring L-α-amino acids. This definition is meant to include norleucine, ornithine, and homocysteine.

As used herein, in general, the term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, and so on.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have one or two amino acids deleted in a particular region of the molecule.

As used herein, “antagonist” refers to a ligand that tends to nullify the action of another ligand, as a ligand that binds to a cell receptor without eliciting a biological response.

It is also contemplated that TOY fusion proteins be labeled with a detectable label, such as radioisotope, fluorescent tag, enzymatic tag, or a chemiluminescent tag to determine ligand-receptor binding interaction. As such, assay systems employing the chimeric molecule are also contemplated.

As used herein, “carriers” include pharmaceutically acceptable carriers, excipients, or stabilizers which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein, “effective amount” is an amount sufficient to effect beneficial or desired clinical or biochemical results. An effective amount can be administered one or more times. For purposes of this invention, an effective amount of an inhibitor compound is an amount that is sufficient to palliate, ameliorate, stabilize, reverse, slow or delay the progression of the disease state.

As used herein, “fragments” or “functional derivatives” refers to biologically active amino acid sequence variants and fragments of the native ligands or receptors of the present invention, as well as covalent modifications, including derivatives obtained by reaction with organic derivatizing agents, post-translational modifications, derivatives with nonproteinaceous polymers, and immunoadhesins.

As used herein, “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change.

As used herein, “ligand” refers to any molecule or agent, or compound that specifically binds covalently or transiently to a molecule such as a polypeptide. When used in certain context, ligand may include antibody. In other context, “ligand” may refer to a molecule sought to be bound by another molecule with high affinity, such as in a ligand trap.

As used herein, “mammal” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

As used herein, “sample” or “biological sample” is referred to in its broadest sense, and includes any biological sample obtained from an individual, body fluid, cell line, tissue culture, or other source which may contain a chimeric Ang1 binding factor, depending on the type of assay that is to be performed. As indicated, biological samples include body fluids, such as semen, lymph, sera, plasma, urine, synovial fluid, spinal fluid and so on. Methods for obtaining tissue biopsies and body fluids from mammals are well known in the art.

As used herein, “subject” is a vertebrate, preferably a mammal, more preferably a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-structures, such as polyamides.

Nucleic Acid Constructs

Also provided is an expression vector comprising a nucleic acid molecule of the invention as described herein, wherein the nucleic acid molecule is operatively linked to an expression control sequence. Also provided is a host-vector system for the production of a fusion polypeptide which comprises the expression vector of the invention which has been introduced into a host cell suitable for expression of the fusion polypeptide. The suitable host cell may be a bacterial cell such as E. coli, a yeast cell, such as Pichia pastoris, an insect cell, such as Spodoptera frugiperda, or a mammalian cell, such as a COS or CHO cell.

The present invention also provides for methods of producing the fusion polypeptides of the invention by growing cells of the host-vector system described herein, under conditions permitting production of the fusion polypeptide and recovering the fusion polypeptide so produced. The fusion polypeptides useful for practicing the present invention may be prepared by expression in a prokaryotic or eukaryotic expression system.

The recombinant gene may be expressed and the polypeptide purified utilizing any number of methods. The gene may be subcloned into a bacterial expression vector, such as for example, but not by way of limitation, pZErO.

The fusion polypeptides may be purified by any technique which allows for the subsequent formation of a stable, biologically active protein. For example, and not by way of limitation, the factors may be recovered from cells either as soluble proteins or as inclusion bodies, from which they may be extracted quantitatively by 8M guanidinium hydrochloride and dialysis. In order to further purify the factors, any number of purification methods may be used, including but not limited to conventional ion exchange chromatography, affinity chromatography, different sugar chromatography, hydrophobic interaction chromatography, reverse phase chromatography or gel filtration.

When used herein, fusion polypeptide includes functionally equivalent molecules in which amino acid residues are substituted for residues within the sequence resulting in a silent or conservative change. For example, one or more amino acid residues within the sequence can be substituted by another amino acid of a similar polarity which acts as a functional equivalent, resulting in a silent or conservative alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, etc.

Cells that express the fusion polypeptides of the invention are genetically engineered to produce them by, for example, transfection, transduction, electroporation, or microinjection techniques.

In addition, the present invention contemplates use of the fusion polypeptides described herein in tagged form.

Any of the methods known to one skilled in the art for the insertion of DNA fragments into a vector may be used to construct expression vectors encoding the fusion polypeptides of the invention using appropriate transcriptional/translational control signals and protein coding sequences. These methods may include in vitro recombinant DNA and synthetic techniques and in vivo recombinations (genetic recombination). Expression of nucleic acid sequence encoding the fusion polypeptides of the invention may be regulated by a second nucleic acid sequence so that the fusion polypeptide is expressed in a host transformed with the recombinant DNA molecule. For example, expression of the fusion polypeptides described herein may be controlled by any promoter/enhancer element known in the art. Promoters which may be used to control expression of the fusion polypeptide include, but are not limited to the long terminal repeat as described in Squinto et al., (1991, Cell 65:1-20); the SV40 early promoter region (Bernoist and Chambon, 1981, Nature 290:304-310), the CMV promoter, the M-MuLV 5′ terminal repeat the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:144-1445), the regulatory sequences of the metallothionein gene (Brinster et al., 1982, Nature 296:39-42); prokaryotic expression vectors such as the β-lactamase promoter (VIIIa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25), see also “Useful proteins from recombinant bacteria” in Scientific American, 1980, 242:74-94; promoter elements from yeast or other fungi such as the Gal 4 promoter, the ADH (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58); alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286), and gonadotropic releasing hormone gene control region which is active in the hypothalamus (Mason et al., 1986, Science 234:1372-1378).

Thus, according to the invention, expression vectors capable of being replicated in a bacterial or eukaryotic host comprising nucleic acids encoding a fusion polypeptide as described herein, and in particular modified TOY, are used to transfect the host and thereby direct expression of such nucleic acid to produce fusion polypeptides which may then be recovered in biologically active form. As used herein, a biologically active form includes a form capable of binding to the relevant receptor and causing a differentiated function and/or influencing the phenotype of the cell expressing the receptor.

Expression vectors containing the nucleic acid inserts can be identified by without limitation, at least three general approaches: (a) DNA-DNA hybridization, (b) presence or absence of “marker” gene functions, and (c) expression of inserted sequences. In the first approach, the presence of foreign nucleic acids inserted in an expression vector can be detected by DNA-DNA hybridization using probes comprising sequences that are homologous to an inserted nucleic acid sequences. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign nucleic acid sequences in the vector. For example, if an efl nucleic acid sequence is inserted within the marker gene sequence of the vector, recombinants containing the insert can be identified by the absence of the marker gene function. In the third approach, recombinant expression vectors can be identified by assaying the foreign nucleic acid product expressed by the recombinant constructs. Such assays can be based, for example, on the physical or functional properties of the nucleic acid product of interest, for example, by binding of a ligand to a receptor or portion thereof which may be tagged with, for example, a detectable antibody or portion thereof or binding to antibodies produced against the protein of interest or a portion thereof.

The fusion polypeptide, in particular modified TOY of the present invention, may be expressed in the host cells transiently, constitutively or permanently.

The invention herein further provides for the development of a fusion polypeptide as a therapeutic agent for the treatment of patients suffering from disorders involving cells, tissues or organs which express the TLR4, MD-2 and CD14. Such molecules may be used in a method of treatment of the human or animal body, or in a method of diagnosis.

Effective doses useful for treating these or other diseases or disorders may be determined using methods known to one skilled in the art (see, for example, Fingl, et al., The Pharmacological Basis of Therapeutics, Goodman and Gilman, eds. Macmillan Publishing Co, New York, pp. 1-46 (1975). Pharmaceutical compositions for use according to the invention include the fusion polypeptides described above in a pharmacologically acceptable liquid, solid or semi-solid carrier, linked to a carrier or targeting molecule (e.g., antibody, hormone, growth factor, etc.) and/or incorporated into liposomes, microcapsules, and controlled release preparation prior to administration in vivo. For example, the pharmaceutical composition may comprise a fusion polypeptide in an aqueous solution, such as sterile water, saline, phosphate buffer or dextrose solution. Alternatively, the active agents may be comprised in a solid (e.g. wax) or semi-solid (e.g. gelatinous) formulation that may be implanted into a patient in need of such treatment. The administration route may be any mode of administration known in the art, including but not limited to intravenously, intrathecally, subcutaneously, intrauterinely, by injection into involved tissue, intraarterially, intranasally, orally, or via an implanted device.

Administration may result in the distribution of the active agent of the invention throughout the body or in a localized area. For example, in some conditions which involve distant regions of the nervous system, intravenous or intrathecal administration of agent may be desirable. In some situations, an implant containing active agent may be placed in or near the lesioned area. Suitable implants include, but are not limited to, gelfoam, wax, spray, or microparticle-based implants.

The present invention also provides for pharmaceutical compositions comprising the fusion polypeptides described herein, in a pharmacologically acceptable vehicle. The compositions may be administered systemically or locally. Any appropriate mode of administration known in the art may be used, including, but not limited to, intravenous, intrathecal, intraarterial, intranasal, oral, subcutaneous, intraperitoneal, or by local injection or surgical implant. Sustained release formulations are also provided for.

Gene Therapy

In a specific embodiment, nucleic acids comprising sequences encoding the chimeric Ang1 polypeptide are administered to prevent vascular leakage, and for therapeutic vasculogenesis, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, nucleic acid sequences may encode a TLR4 or TLR4/VLRB.61 hybrid polypeptide, in which the nucleic acid sequences are part of expression vectors that express the polypeptides in a suitable host. In particular, such nucleic acid sequences have promoters operably linked to the polypeptide coding region, said promoter being inducible or constitutive, and, optionally, tissue-specific. In another particular embodiment, nucleic acid molecules are used in which the polypeptide coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors, or by direct injection of naked DNA, or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors) and so on. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding the polypeptide are used. The nucleic acid sequences encoding the polypeptide to be used in gene therapy are cloned into one or more vectors, which facilitates delivery of the gene into a patient. Retroviral vectors, adenoviral vectors and adeno-associated viruses are examples of viral vectors that may be used. Retroviral vectors contain the components necessary for the correct packaging of the viral genome and integration into the host cell DNA.

Adenoviruses are especially attractive vehicles for delivering genes to respiratory epithelia because they naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. In addition, adeno-associated virus (AAV) has also been proposed for use in gene therapy.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion and so on. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and so on.

In a preferred embodiment, the cell used for gene therapy is autologous to the patient.

In an embodiment in which recombinant cells are used in gene therapy, nucleic acid sequences encoding the polypeptide are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Therapeutic Composition

In one embodiment, the present invention relates to treatment for various diseases that are characterized by sepsis and septic shock. In this way, the inventive therapeutic compound may be administered to human patients who are either suffering from, or prone to suffer from the disease by providing compounds that activate TLR4.

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitized in vitro and adoptively transferred to the recipient). Depending on the route of administration, the peptide may be required to be coated in a material to protect it from the action of enzymes, acids and other natural conditions which may inactivate said ingredients.

For example, the low lipophilicity of the peptides will allow them to be destroyed in the gastrointestinal tract by enzymes capable of cleaving peptide bonds and in the stomach by acid hydrolysis. In order to administer peptides by other than parenteral administration, they will be coated by, or administered with, a material to prevent its inactivation. For example, peptides may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, thus requiring only a fraction of the systemic dose.

Labels

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H), indium (¹¹²In), and technetium (^{99m}Tc), and fluorescent labels, such as fluorescein and rhodamine, and biotin.

Further suitable labels for the TLR4, MD-2 or TLR4/MD-2 complex-specific antibodies of the present invention are provided below. Examples of suitable enzyme labels include malate dehydrogenase, δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is preferred isotope where in vivo imaging is used since its avoids the problem of dehalogenation of the ¹²⁵I or ¹³¹I-labeled polypeptide by the liver. In addition, this radionucleotide has a more favorable gamma emission energy for imaging. For example, ¹¹¹In coupled to monoclonal antibodies with 1-(P-isothiocyanatobenzyl)-DPTA has shown little uptake in non-tumors tissues, particularly the liver, and therefore enhances specificity of tumor localization.

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include, Pseudomonas toxin, diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron. Deuterium may also be used. Other contrasting agents also exist for EPR, PET or other imaging mechanisms, which are known to persons of skill in the art.

Typical techniques for binding the above-described labels to polypeptides are provided by Kennedy et al. (1976) Clin. Chim. Acta 70:1-31, and Schurs et al. (1977) Clin. Chim. Acta 81:1-40. Coupling techniques include the glutaraldehyde method, the periodate method, the dimaleimide method, the m-maleimidobenzoyl-N-hydroxy-succinimide ester method, all of which methods are incorporated by reference herein.

The polypeptides and antibodies of the present invention, including fragments thereof, may be used to detect TLR4, MD-2, MD-2/LPS complex using biochip and biosensor technology. Biochip and biosensors of the present invention may comprise the polypeptides of the present invention to detect antibodies, which specifically recognize TLR4/MD-2/LPS complex. Bio chip and biosensors of the present invention may also comprise antibodies which specifically recognize the polypeptides of the present invention to detect chimeric TLR4/MD-2/LPS complex.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Gene Construction and Expression of Recombinant TOY Proteins

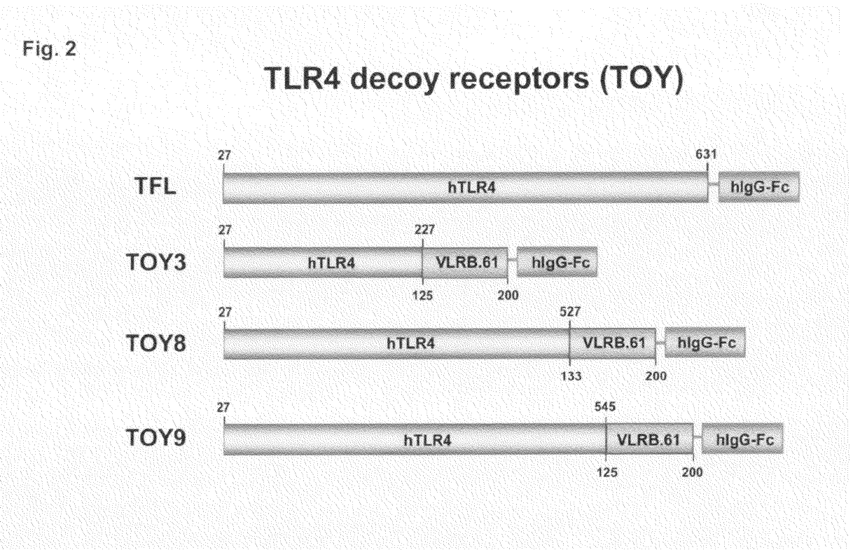

Gene constructs encoding four different assembled fusion proteins (TFL, TOY3, TOY8, and TOY9) (FIG. 2), which include the ectodomain of human TLR4, LRR module of hagfish VLR-B.61 and Fc domain of human IgG were cloned into the pCMV-dhfr vector (Hwang S J, et al., Protein Express Purif., 2005; 39:175-183).

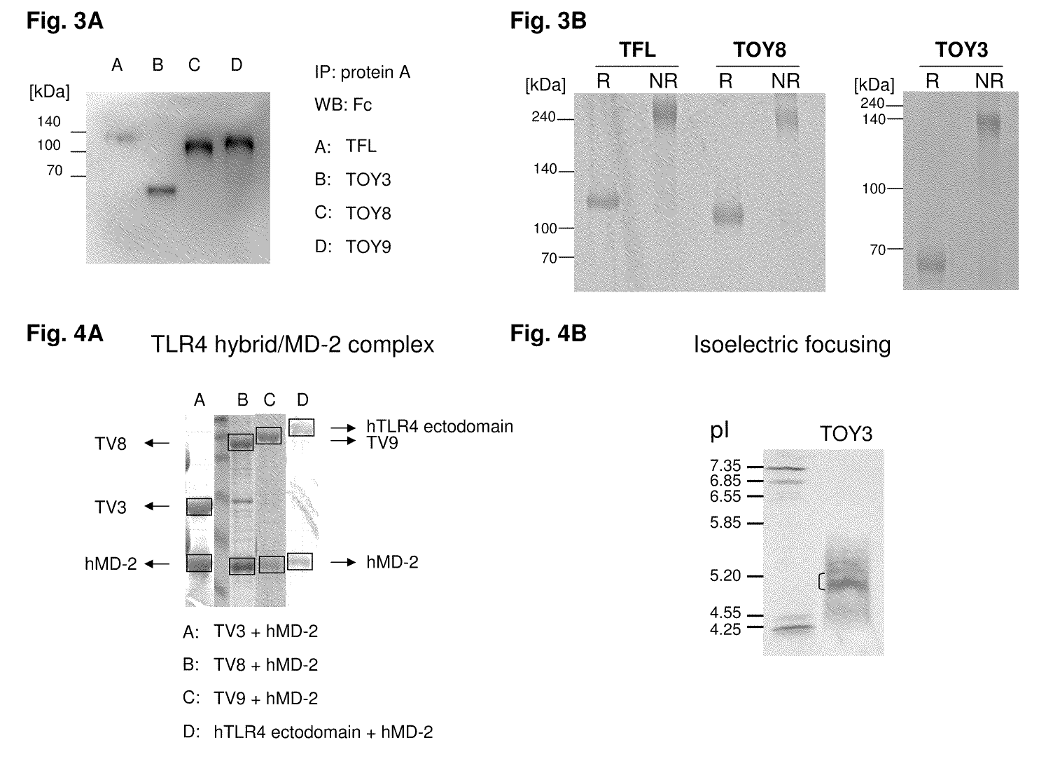

For the Western blotting analysis, hIgG-Fc-fused TFL and each TOY protein from the supernatants of transiently transfected HEK 293 cells were immunoprecipitated with a protein A bead. Each sample was mixed with sample buffer, heat-denatured for 10 min, run on 10% SDS-PAGE, and electro-blotted onto nitrocellulose membranes. The membrane was blocked with 5% nonfat milk in Tris-buffer solution (50 mM Tris, 100 mM NaCl, pH 7.5) containing 0.05% TritonX-100 and Western blotted with horseradish-peroxidase (HRP)-conjugated goat anti-human Fc antibody (1:10,000 dilution; Sigma-Aldrich A0170) to detect Fc-fused proteins. Signal was visualized by chemiluminescent detection according to the manufacturer's protocol (Amersham Pharmacia Biotech) using chemiluminescence scanner (LAS-1000, Fuji Film, Tokyo) (FIG. 3A).

### Example 2

### Mass Production of TOY Proteins Using CHO Cells

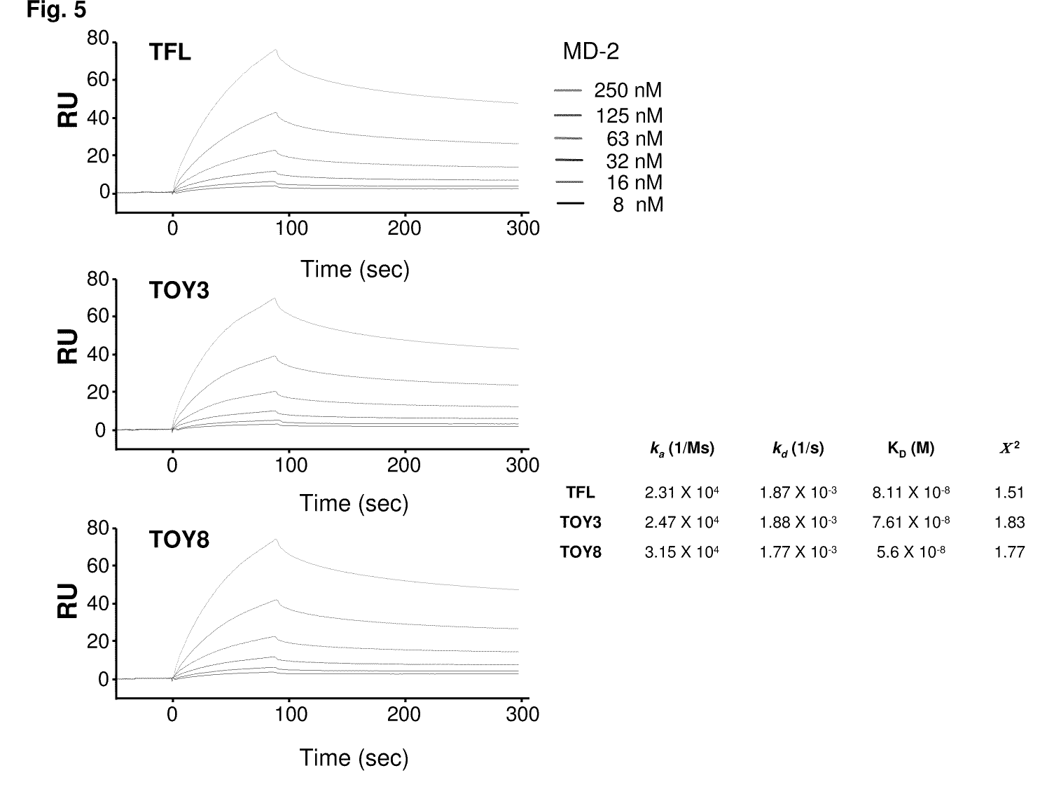

Because all recombinant TOY proteins could interact with MD-2 protein in vitro (FIG. 4A and FIG. 5), they were purified in mammalian system using CHO cells for further studies including in vivo mice experiments.

Recombinant Chinese hamster ovary (rCHO) cells expressing TFL, TOY3, and TOY8 (CHO-TFL, CHO-TOY3, CHO-TOY8, respectively) were established following a previously described method (Hwang S J, et al., Protein Express Purif, 2005; 39:175-183). Briefly, CHO-TFL cells were established by transfection of a vector containing the dihydrofolate reductase (dhfr) and TFL genes into dhfr-deficient CHO cells (CRL-9096, American Type Culture Collection, Manassas, Va., USA). This was followed by dhfr/methotrexate (MTX)-mediated gene amplification. The five stable rCHO cells secreting TFL were selected with serial amplified concentrations of MTX (0.001-1.0 μM, Sigma-Aldrich). Among them, one cell line expressing the highest amount of TFL was chosen and named as “CHO-TFL”. CHO-TFL cells were grown and maintained in Iscove's modified Dulbecco's medium supplemented with 5% dialyzed fetal bovine serum (Invitrogen, Carlsbad, Calif., USA) and 1 μM MTX (Sigma-Aldrich). For recombinant TFL protein production, CHO-TFL cells were inoculated at 2×10⁵ cells/mL in 250-ml Erlenmeyer flasks containing 100 ml of medium on an orbital shaker (Vision, Bucheon, Korea) at 110 rpm in a humidified 5% CO₂ incubator at 37° C. After four days, the culture medium containing recombinant TFL proteins were harvested and the recombinant TFL proteins were purified by using Protein-A sepharose affinity chromatography with subsequent acid elution and neutralization. After purification, the protein was quantitated using the Bradford assay and confirmed with Coomassie blue staining of an SDS-PAGE gel. The same procedures were applied for production of TOY3 and TOY8 (FIG. 3B).

### Example 3

### MD-2 Binding Capability and Isoelectric Focusing of the TOY Proteins

Protein A-tagged MD-2 and the TV3, TV8, TV9, or hTLR4 ectodomain were co-infected into insect cells, subsequently purified using IgG Sepharose (GE Healthcare) affinity chromatography, and analyzed under SDS-PAGE (FIG. 4A). Each of the TV hybrids or hTLR4 ectodomain and MD-2 proteins formed a stable 1:1 complex and were not separated during purification. Therefore, all recombinant TLR4/VLR-B.61 fusion hybrid proteins were capable of binding MD-2.

Theoretical pI value and molecular weight of each recombinant protein is 5.7, 94 kDa for TFL, 11.8, 53 kDa for TOY3, 6.0, 90 kDa for TOY8, and 6.0, 93 kDa for TOY9. TOY3 bearing only the A patch of the TLR4 without the B patch showed relatively high pI value compared to other TOY proteins. Virtual isoelectric point value of TOY3 produced in CHO cells was analyzed with IsoGel Agarose IEF plate (Cambrex, N.J., USA). TOY3 resulted in pI value of about 5.2, much lower than its theoretical value, which would be mainly due to abundant glycosylation process in CHO cells (FIG. 4B).

### Example 4

### Analysis of the Interaction Between Each TOY and MD-2

The interactions for binding between TFL and MD-2, between TOY3 and MD-2, and between TOY8 and MD-2 were analyzed with BIAcore 3000 (BIAcore AB, Uppsala, Sweden). TFL, TOY3 or TOY8 (30 μg/ml) in 10 mM sodium acetate (pH 5.5) was immobilized on a CM5 sensor chip using the amine-coupling method. MD-2 in HBS-EP buffer (BIAcore AB) at a concentration of 8 nM to 250 nM was passed over the surface of the sensor chip at a flow rate of 60 μl/min. The interactions were monitored as the changes of surface plasmon resonance response at 25° C. After 1 min of monitoring, the same buffer was introduced onto the sensor chip in place of the MD-2 solution to start the dissociation. The sensor surface was regenerated with 10 mM Glycine (pH 2.3) at the end of each experiment. Both the association rate constant (kₐ) and the dissociation rate constant (k_{d}) were calculated according to the BIAevaluation software (version 3.1; BIAcore AB) using a program named 1:1 (Langmuir) binding model. The dissociation constant (K_{D}) was determined by kₐ/k_{d}.

These analyses for those interactions revealed that K_{D} of TFL to MD-2 was ˜81 nM and that of TOY3 to MD-2 was ˜76 nM, whereas K_{D} of TOY8 to MD-2 was ˜56 nM (FIG. 5). Therefore, fusion of the VLRB.61 component for recombinant TLR4 protein did not alter the binding affinity of the intrinsic native TLR4 protein not bearing VLRB.61 portion.

### Example 5

### Preventive Effect of TOY3 in LPS-Induced Sepsis in a Mouse Model

To examine the in vivo preventive effect of TOY3 protein on sepsis, lethality studies using LPS-induced sepsis in a mouse model were performed. Eight-week-old male C³H/HeN mice were injected with 15 mg/kg of LPS (E. coli O111:B4; List Biological Laboratories, Campbell, Calif.) into the peritoneal cavity and monitored for survival. Ten minutes before LPS administration, mice received 20 mg/kg of Fc (n=5) or TOY3 (n=6) into the peritoneal cavity.

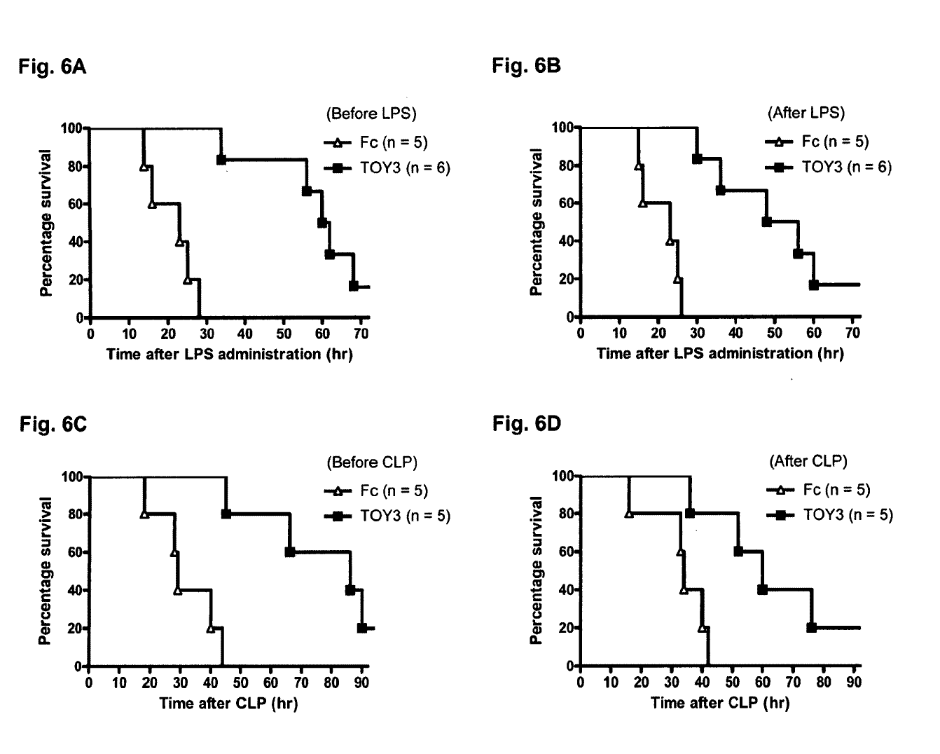

In Fc-treated group, 40% of the mice died within 22 hours after LPS administration and the rest of 60% died within 28 hours. However, in TOY3-treated group, none of the mice died within 32 hours, and more than 80% of the mice were alive at 55 hours. Furthermore, about 20% of the mice were still alive by 70 hours after LPS treatment. This result explains that TOY3-treated mice showed prolonged lifespan compared to Fc-treated mice, therefore, TOY3 has prominent preventive effect in LPS-induced sepsis in a mouse model (TOY3 versus Fc, P<0.001) (FIG. 6A).

### Example 6

### Therapeutic Effect of TOY3 in LPS-Induced Sepsis in a Mouse Model

To examine the in vivo therapeutic effect of TOY3 protein on sepsis, similar lethality studies using LPS-induced sepsis in a mouse model were performed. Eight-week-old male C³H/HeN mice were injected with 15 mg/kg of LPS (E. coli O111:B4; List Biological Laboratories, Campbell, Calif.) into the peritoneal cavity and monitored for survival. One hour after LPS administration, mice received 20 mg/kg of Fc (n=5), TOY3 (n=6) into the peritoneal cavity.

In Fc-treated group, 40% of the mice died within 16 hours and the rest were dead within 27 hours after LPS administration. However, in TOY3-treated group, none of the mice died within 30 hours, and one half of the mice were alive at 55 hours after LPS administration. Surprisingly, about 20% of the TOY3-treated mice were still alive at 70 hours after LPS treatment. This result shows that TOY3 also has prominent therapeutic effect in LPS-induced sepsis in a mouse model (TOY3 versus Fc, P<0.001). Also, this experiment implies that the A patch of the TLR4 ectodomain is sufficient for the MD-2 binding in vivo, because TOY3 bearing only the A patch showed significant effect on treatment of sepsis (FIG. 6B). This result is also consistent with the result of binding affinity analysis using BIAcore (FIG. 5).

### Example 7

### Preventive Effect of TOY3 in CLP-Induced Sepsis in a Mouse Model

To examine the in vivo preventive effect of TOY3 protein on sepsis, lethality studies using another sepsis in a mouse model, cecal ligation and puncture (CLP) model, were performed. Mice were anesthetized with 80 mg/kg of ketamine and 12 mg/kg of xylazine. After shaving the abdomen, a 2-cm midline incision was created under aseptic conditions to expose the cecum and adjoining intestine. Approximately 75% of the cecum was ligated distal to the ileo-cecal valve with 4-0 vicryl suture and punctured with 21-gauge needle. The cecum was then gently squeezed to extrude a small amount of feces to ensure patency of the perforation sites and was returned to the peritoneal cavity. One hour before CLP procedure, mice received 20 mg/kg of Fc (n=5) or TOY3 (n=5) into the peritoneal cavity and the mice were monitored for survival.

In Fc-treated group, 60% of the mice died within 29 hours and none of the mice were alive at 43 hours after CLP procedure. Meanwhile, in TOY3-treated group, only 20% of the mice died within 44 hours, and 40% were still alive at 84 hours after CLP procedure. Finally, 20% of the mice were normalized. This result shows that TOY3-treated mice showed prolonged lifespan compared to Fc-treated mice (P<0.005), therefore, TOY3 also has preventive effect even in CLP-induced sepsis in a mouse model (FIG. 6C).

### Example 8

### Therapeutic Effect of TOY3 in CLP-Induced Sepsis in a Mouse Model

To examine the in vivo therapeutic effect of TOY3 protein on sepsis, similar lethality studies using cecal ligation and puncture (CLP) model were performed as described above. One hour after CLP procedure, mice received 20 mg/kg of Fc (n=5) or TOY3 (n=5) into the peritoneal cavity and the mice were monitored for survival.

In Fc-treated group, 60% of the mice died within 33 hours and the rest died within 42 hours after CLP procedure. Meanwhile, in TOY3-treated group, only 20% of the mice died within 37 hours, and 40% were still alive at 75 hours after CLP procedure. Also, 20% of the mice were finally normalized. Taken together, TOY3 has not only preventive but also therapeutic effect in CLP-induced sepsis in a mouse model compared to control Fc (P<0.05) (FIG. 6D).

### Example 9

### Therapeutic Effect of TOY3 on Improving Organ Damage in LPS-Induced Sepsis in a Mouse Model

To examine the in vivo effect of TOY3 protein on sepsis-induced organ damage in LPS-induced sepsis model, mice were injected with 8 mg/kg of LPS (E. coli O111:B4; List Biological Laboratories, Campbell, Calif.) into the peritoneal cavity. One hour after LPS administration, mice received 20 mg/kg of Fc or TOY3 into the peritoneal cavity. Mice were sacrificed 24 hours after LPS treatment, and their livers and lungs were harvested for histology studies. Prepared organs were fixed in 4% PFA in 4° C. overnight and embedded in paraffin blocks. Four-micrometer sections were stained with H&E staining and analyzed under phase-contrast light microscope.

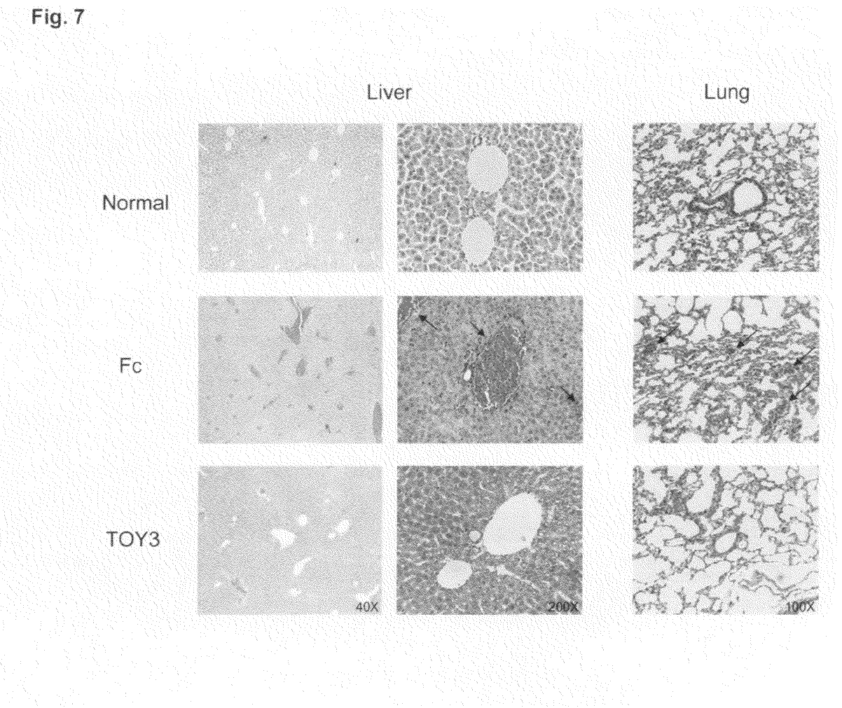

Formation of thrombi in blood vessel is one of the principal indications of injured organs in sepsis. In livers, typical portal tract regions were focused and analyzed to compare the formation of thrombi. Almost all blood vessels in liver were obstructed caused by thrombosis in Fc-treated mice. High-magnification analysis showed that many blood vessels were filled with red blood cells (RBCs). In contrast, the number of thrombi in liver of TOY3-treated mice was significantly lower than that of Fc-treated mice. There were only few RBCs accumulated in many vessels in TOY3-treated mice similar to normal control mice. To examine injuries in a more distant organ, lungs were also analyzed. In lung of Fc-treated mice, a large number of intravascular thrombi were observed around alveoli structures. However, presence of intravascular thrombi in lung of TOY3-treated mice was hardly detected compared to that of Fc-treated mice (FIG. 7). Therefore, TOY3 has therapeutic effect on improving, treating or preventing organ damage in LPS-induced sepsis in a mouse model in vivo.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

TABLE 1

| TFL | |
|---|---|
| ATG CTA CTA GTA AAT CAG TCA CAC CAA GGC TTC AAT AAG GAA CAC ACA AGC AAG ATG GTA | (SEQ ID NO: 1) |
| TAC GAT GAT CAT TTA GTC AGT GTG GTT CCG AAG TTA TTC CTT GTG TGT TCG TTC TAC CAT | |
| Met Leu Leu Val Asn Gln Ser His Gln Gly Phe Asn Lys Glu His Thr Ser Lys Met Val | (SEQ ID NO: 2) |
| 1 5 gp67 secretion signal sequence 15 20 | |
| AGC GCT ATT GTT TTA TAT GTG CTT TTG GCG GCG GCG GCG CAT TCT GCC TTT GCG GCG GAT | |
| TCG CGA TAA CAA AAT ATA CAC GAA AAC CGC CGC CGC CGC GTA AGA CGG AAA CGC CGC CTA | |
| Ser Ala Ile Val Leu Tyr Val Leu Leu Ala Ala Ala Ala His Ser Ala Phe Ala Ala Asp | |
| 21 25 gp67 secretion signal sequence 35 38 40 | |
| CCC GAG CCC TGC GTG GAG GTG GTT CCT AAT ATT ACT TAT CAA TGC ATG GAG CTG AAT TTC | |
| GGG CTC GGG ACG CAC CTC CAC CAA GGA TTA TAA TGA ATA GTT ACG TAC CTC GAC TTA AAG | |
| Pro Glu Pro Cys Val Glu Val Val Pro Asn Ile Thr Tyr Gln Cys Met Glu Leu Asn Phe | |
| 41 42 45 LRRNT 55 60 | |
| TAC AAA ATC CCC GAC AAC CTC CCC TTC TCA ACC AAG AAC CTG GAC CTG AGC TTT AAT CCC | |
| ATG TTT TAG GGG CTG TTG GAG GGG AAG AGT TGG TTC TTG GAC CTG GAC TCG AAA TTA GGG | |
| Tyr Lys Ile Pro Asp Asn Leu Pro Phe Ser Thr Lys Asn Leu Asp Leu Ser Phe Asn Pro | |
| 61 LRRNT 66 67 LRR1 80 | |
| CTG AGG CAT TTA GGC AGC TAT AGC TTC TTC AGT TTC CCA GAA CTG CAG GTG CTG GAT TTA | |
| GAC TCC GTA AAT CCG TCG ATA TCG AAG AAG TCA AAG GGT CTT GAC GTC CAC GAC CTA AAT | |
| Leu Arg His Leu Gly Ser Tyr Ser Phe Phe Ser Phe Pro Glu Leu Gln Val Leu Asp Leu | |
| 81 LRR1 90 91 LRR2 100 | |
| TCC AGG TGT GAA ATC CAG ACA ATT GAA GAT GCG GCA TAT CAG AGC CTA AGC CAC CTC TCT | |
| AGG TCC ACA CTT TAG GTC TGT TAA CTT CTA CCC CGT ATA GTC TCG GAT TCG GTG GAG AGA | |
| Ser Arg Cys Glu Ile Gln Thr Ile Glu Asp G1y Ala Tyr Gln Ser Leu Ser His Leu Ser | |
| 101 LRR2 110 113 114 LRR3 120 | |
| ACC TTA ATA TTG ACA GGA AAC CCC ATC CAG AGT TTA GCC CTG GGA GCC TTT TCT GGA CTA | |
| TGG AAT TAT AAC TGT CCT TTG GGG TAG GTC TCA AAT CGG GAC CCT CGG AAA AGA CCT GAT | |
| Thr Leu Ile Leu Thr Gly Asn Pro Ile Gln Ser Leu Ala Leu Gly Ala Phe Ser Cly Leu | |
| 121 125 LRR3 138 139 140 | |
| TCA AGT TTA CAG AAG CTG GTG GCT GTG GAG ACA AAT CTA GCA TCT CTA GAG AAC TTC CCC | |
| AGT TCA AAT GTC TTC GAC CAC CGA CAC CTC TGT TTA GAT CGT AGA GAT CTC TTG AAG GGG | |
| Ser Ser Leu Gln Lys Leu Val Ala Val Glu Thr Asn Leu Ala Ser Leu Glu Asn Phe Pro | |
| 141 145 LRR4 155 160 | |
| ATT GGA CAT CTC AAA ACT TTG AAA GAA CTT AAT GTG GCT CAC AAT CTT ATC CAA TCT TTC | |
| TAA CCT GTA GAG TTT TGA AAC TTT CTT GAA TTA CAC CGA GTG TTA GAA TAG GTT AGA AAG | |
| Ile Gly His Leu Lys Thr Leu Lys Glu Leu Asn Val Ala His Asn Leu Ile Gln Ser Phe | |
| 161 162 163 LRR5 175 180 | |
| AAA TTA CCT GAG TAT TTT TCT AAT CTG ACC AAT CTA GAG CAC TTG GAC CTT TCC AGC AAC | |
| TTT AAT GGA CTC ATA AAA AGA TTA GAC TGG TTA GAT CTC GTG AAC CTG GAA AGG TCG TTG | |
| Lys Leu Pro Glu Tyr Phe Ser Asn Leu Thr Asn Leu Glu His Leu Asp Leu Ser Ser Asn | |
| 181 LRR5 187 188 LRR6 200 | |
| AAG ATT CAA AGT ATT TAT TGC ACA GAC TTG CGG GTT CTA CAT CAA ATG CCC CTA CTC AAT | |
| TTC TAA GTT TCA TAA ATA ACG TGT CTG AAC GCC CAA GAT GTA GTT TAC GGG GAT GAG TTA | |
| Lys Ile Gln Ser Ile Tyr Cys Thr Asp Leu Arg Val Leu His Gln Met Pro Leu Leu Asn | |
| 201 205 LRR6 217 218 220 | |
| CTC TCT TTA GAC CTG TCC CTG AAC CCT ATG AAC TTT ATC CAA CCA GGT GCA TTT AAA GAA | |
| GAG AGA AAT CTG GAC AGG GAC TTG GGA TAC TTG AAA TAG GTT GGT CCA CGT AAA TTT CTT | |
| Leu Ser Leu Asp Leu Ser Leu Asn Pro Met Asn Phe Ile Gln Pro Gly Ala Phe Lys Glu | |
| 221 225 LRR7 235 239 240 | |
| ATT AGG CTT CAT AAG CTG ACT TTA AGA AAT AAT TTT GAT AGT TTA AAT GTA ATG AAA ACT | |
| TAA TCC GAA GTA TTC GAC TGA AAT TCT TTA TTA AAA CTA TCA AAT TTA CAT TAC TTT TGA | |
| Ile Arg Leu His Lys Leu Thr Leu Arg Asn Asn Phe Asp Ser Leu Asn Val Met Lys Thr | |
| 241 245 LRR8 255 260 | |
| TGT ATT CAA GGT CTG GCT GGT TTA GAA GTC CAT CGT TTG GTT CTG GGA GAA TTT AGA AAT | |
| ACA TAA GTT CCA GAC CGA CCA AAT CTT CAG GTA GCA AAC CAA GAC CCT CTT AAA TCT TTA | |
| Cys Ile Gln Gly Leu Ala Gly Leu Glu Val His Arg Leu Val Leu Gly Glu The Arg Asn | |
| 261 LRR8 266 267 LRR9 280 | |
| GAA GGA AAC TTG GAA AAG TTT GAC AAA TCT GCT CTA GAG GGC CTG TGC AAT TTG ACC ATT | |
| CTT CCT TTG AAC CTT TTC AAA CTG TTT AGA CGA GAT CTC CCG GAC ACG TTA AAC TGG TAA | |
| Glu Gly Asn Leu Glu Lys Phe Asp Lys Ser Ala Leu Glu Gly Leu Cys Asn Leu Thr Ile | |
| 281 285 LRR9 296 297 LRR10 300 | |
| GAA GAA TTC CGA TTA GCA TAC TTA GAC TAC TAC CTC GAT GAT ATT ATT GAC TTA TTT AAT | |
| CTT CTT AAG GCT AAT CGT ATG AAT CTG ATG ATG GAG CTA CTA TAA TAA CTG AAT AAA TTA | |
| Glu Glu Phe Arg Leu Ala Tyr Leu Asp Tyr Tyr Leu Asp Asp Ile Ile Asp Leu Phe Asn | |
| 301 305 LRR10 315 320 | |
| TGT TTG ACA AAT GTT TCT TCA TTT TCC CTG GTG AGT GTG ACT ATT GAA AGG GTA AAA GAC | |
| ACA AAC TGT TTA CAA AGA AGT AAA AGG GAC CAC TCA CAC TGA TAA CTT TCC CAT TTT CTG | |
| Cys Leu Thr Asn Val Ser Ser Phe Ser Leu Val Ser Val Thr Ile Glu Arg Val Lys Asp | |
| 321 325 LRR11 335 340 | |
| TTT TCT TAT AAT TTC GGA TGG CAA CAT TTA GAA TTA GTT AAC TGT AAA TTT GGA CAG TTT | |
| AAA AGA ATA TTA AAG CCT ACC GTT GTA AAT CTT AAT CAA TTG ACA TTT AAA CCT GTC AAA | |
| Phe Ser Tyr Asn Phe Gly Trp Gln His Leu Glu Leu Val Asn Cys Lys Phe Gly Gln Phe | |
| 341 343 344 LRR12 355 360 | |
| CCC ACA TTG AAA CTC AAA TCT CTC AAA AGG CTT ACT TTC ACT TCC AAC AAA GGT GGG AAT | |
| GGG TGT AAC TTT GAG TTT AGA GAG TTT TCC GAA TGA AAG TGA AGG TTG TTT CCA CCC TTA | |
| Pro Thr Leu Lys Leu Lys Ser Leu Lys Arg Leu Thr Phe Thr Ser Asn Lys Gly Gly Asn | |
| 361 363 364 LRR13 375 380 | |
| GCT TTT TCA GAA GTT GAT CTA CCA AGC CTT GAG TTT CTA GAT CTC AGT AGA AAT GGC TTG | |
| CGA AAA AGT CTT CAA CTA GAT GGT TCG GAA CTC AAA GAT CTA GAG TCA TCT TTA CCG AAC | |
| Ala Phe Ser Glu Val Asp Leu Pro Ser Leu Glu Phe Leu Asp Leu Ser Arg Asn Gly Leu | |
| 381 LRR13 385 386 LRR14 395 400 | |
| AGT TTC AAA GGT TGC TGT TCT CAA AGT GAT TTT GGG ACA ACC AGC CTA AAG TAT TTA GAT | |
| TCA AAG TTT CCA ACG ACA AGA GTT TCA CTA AAA CCC TGT TGG TCG GAT TTC ATA AAT CTA | |
| Ser Phe Lys Gly Cys Cys Ser Gln Ser Asp Phe Gly Thr Thr Ser Leu Lys Tyr Leu Asp | |
| 401 LRR14 411 412 LRR15 420 | |
| CTG AGC TTC AAT GGT GTT ATT ACC ATG AGT TCA AAC TTC TTG GGC TTA GAA CAA CTA GAA | |
| GAC TCG AAG TTA CCA CAA TAA TGG TAC TCA AGT TTG AAG AAC CCG AAT CTT GTT GAT CTT | |
| Leu Ser Phe Asn Gly Val Ile Thr Met Ser Ser Asn Phe Leu Gly Leu Glu Gln Leu Glu | |
| 421 LRR15 434 435 LRR16 440 | |
| CAT CTG GAT TTC CAG CAT TCC AAT TTG AAA CAA ATG AGT GAG TTT TCA GTA TTC CTA TCA | |
| GTA GAC CTA AAG GTC GTA AGG TTA AAC TTT GTT TAC TCA CTC AAA AGT CAT AAG GAT AGT | |
| His Leu Asp Phe Gln His Ser Asn Leu Lys Gln Met Ser Glu Phe Ser Val Phe Leu Ser | |
| 441 LRR16 459 460 | |
| CTC AGA AAC CTC ATT TAC CTT GAC ATT TCT CAT ACT CAC ACC AGA GTT GCT TTC AAT GGC | |
| GAG TCT TTG GAG TAA ATG GAA CTG TAA AGA GTA TGA GTG TGG TCT CAA CGA AAG TTA CCG | |
| Leu Arg Asn Leu Ile Tyr Leu Asp Ile Ser His Thr His Thr Arg Val Ala Phe Asn Gly | |
| 461 465 LRR17 475 480 | |
| ATC TTC AAT GGC TTG TCC AGT CTC GAA GTC TTG AAA ATG GCT GGC AAT TCT TTC CAG GAA | |
| TAG AAG TTA CCG AAC AGG TCA GAG CTT CAG AAC TTT TAC CGA CCG TTA AGA AAG GTC CTT | |
| Ile Phe Asn Gly Leu Ser Ser Leu Glu Val Leu Lys Met Ala Gly Asn Ser Phe Gln Glu | |
| 481 483 484 LRR18 495 500 | |
| AAC TTC CTT CCA GAT ATC TTC ACA GAG CTG AGA AAC TTG ACC TTC CTG GAC CTC TCT CAG | |
| TTG AAG GAA GGT CTA TAG AAG TGT CTC GAC TCT TTG AAC TGG AAG GAC CTG GAG AGA GTC | |
| Asn Phe Leu Pro Asp Ile Phe Thr Glu Leu Arg Asn Leu Thr Phe Leu Asp Leu Ser Gln | |
| 501 LRR18 508 509 LRR19 520 | |
| TGT CAA CTG GAG CAG TTG TCT CCA ACA GCA TTT AAC TCA CTC TCC AGT CTT CAG GTA CTA | |
| ACA GTT GAC CTC GTC AAC AGA GGT TGT CGT AAA TTG AGT GAG AGG TCA GAA GTC CAT GAT | |
| Cys Gln Leu Glu Gln Leu Ser Pro Thr Ala Phe Asn Ser Leu Ser Ser Leu Gln Val Leu | |
| 521 LRR19 532 533 LRR20 540 | |
| AAT ATG AGC CAC AAC AAC TTC TTT TCA TTG GAT ACG TTT CCT TAT AAG TGT CTG AAC TCC | |
| TTA TAC TCG GTG TTG TTG AAG AAA AGT AAC CTA TGC AAA GGA ATA TTC ACA GAC TTG AGG | |
| Asn Met Ser His Asn Asn Phe Phe Ser Leu Asp Thr Phe Pro Tyr Lys Cys Leu Asn Ser | |
| 541 LRR20 550 555 557 560 | |
| CTC AAA GAG CTC GCC CTG GAC ACC AAC CAG CTG AAG TCT GTT CCT GAT GGG ATT TTT GAT | |
| GAG TTT CTC GAG CGG GAC CTG TGG TTG GTC GAC TTC AGA CAA GGA CTA CCC TAA AAA CTA | |
| Leu Lys Glu Leu Ala Leu Asp Thr Asn Gln Leu Lys Ser Val Pro Asp Gly Ile Phe Asp | |
| 561 LRR21 570 580 | |
| CGC CTG ACC AGC TTG CAG AAA ATT TGG CTT CAT ACA AAC CCT TGG GAC TGC AGT TGT CCC | |
| GCG GAC TGG TCG AAC GTC TTT TAA ACC GAA GTA TGT TTG GGA ACC CTG ACG TCA ACA GGG | |
| Arg Leu Thr Ser Leu Gln Lys Ile Trp Leu His Thr Asn Pro Trp Asp Cys Ser Cys Pro | |
| 581 LRR21 590 600 | |
| AGG ATT GAC TAT CTC AGT CGC TGG TTA AAT AAA AAC TCA CAG AAG GAA CAG GGC AGT GCT | |
| TCC TAA CTG ATA GAG TCA GCG ACC AAT TTA TTT TTG AGT GTC TTC CTT GTC CCG TCA CGA | |
| Arg Ile Asp Tyr Leu Ser Arg Trp Leu Asn Lys Asn Ser Gln Lys Glu Gln Gly Ser Ala | |
| 601 LRR22 610 620 | |
| AAA TGT TCC GGG TCC GGC AAG CCC GTC CGA AGT ATC ATC TGC CCT ACT CTC GAG GAC AAA | |
| TTT ACA AGG CCC AGG CCG TTC GGG CAG GCT TCA TAG TAG ACG GGA TGA GAG CTC CTG TTT | |
| Lys Cys Ser Gly Ser Gly Lys Pro Val Arg Ser Ile Ile Cys Pro Thr Leu Glu Asp Lys | |
| 621 LRR22 630 636 637 hFC DOMAIN_{—} | |
| ACT CAC ACA TGC CCA CCG TGC CCA GCA CCT GAA CTC CTG GGG GGA CCG TCA GTC TTC CTC | |
| TGA GTG TGT ACG GGT GGC ACG GGT CGT GGA CTT GAG GAC CCC CCT GGC AGT CAG AAG GAG | |
| Thr His Thr Cys Pro Pro Cys Pro Ala Pro Glu Leu Leu Gly Gly Pro Ser Val Phe Leu | |
| 641 645 hFC DOMAIN 655 660 | |
| TTC CCC CCA AAA CCC AAG GAC ACC CTC ATG ATC TCC CGG ACC CCT GAG GTC ACA TGC GTG | |
| AAG GGG GGT TTT GGG TTC CTG TGG GAG TAC TAG AGG GCC TGG GGA CTC CAG TGT ACG CAC | |
| Phe Pro Pro Lys Pro Lys Asp Thr Leu Met Ile Ser Arg Thr Pro Glu Val Thr Cys Val | |
| 661 665 hFC DOMAIN 675 680 | |
| GTG GTG GAC GTG AGC CAC GAA GAC CCT GAG GTC AAG TTC AAC TGG TAC GTG GAC GGC GTG | |
| CAC CAC CTG CAC TCG GTG CTT CTG GGA CTC CAG TTC AAG TTG ACC ATG CAC CTG CCG CAC | |
| Val Val Asp Val Ser His Glu Asp Pro Glu Val Lys Phe Asn Trp Tyr Val Asp Gly Val | |
| 681 685 hFC DOMAIN 695 700 | |
| GAG GTG CAT AAT GCC AAG ACA AAG CCG CGG GAG GAG CAG TAC AAC AGC ACG TAC CGT GTG | |
| CTC CAC GTA TTA CGG TTC TGT TTC GGC GCC CTC CTC GTC ATG TTG TCG TGC ATG GCA CAC | |
| Glu Val His Asn Ala Lys Thr Lys Pro Arg Glu Glu Gln Tyr Asn Ser Thr Tyr Arg Val | |
| 701 705 hFC DOMAIN 715 720 | |
| GTC AGC GTC CTC ACC GTC CTG CAC CAG GAC TGG CTG AAT GGC AAG GAG TAC AAG TGC AAG | |
| CAG TCG CAG GAG TGG CAG GAC GTG GTC CTG ACC GAC TTA CCG TTC CTC ATG TTC ACG TTC | |
| Val Ser Val Leu Thr Val Leu His Gln Asp Trp Leu Asn Gly Lys Glu Tyr Lys Cys Lys | |
| 721 725 hFC DOMAIN 735 740 | |
| GTC TCC AAC AAA GCC CTC CCA GCC CCC ATC GAG AAA ACC ATC TCC AAA GCC AAA GGG CAG | |
| CAG AGG TTG TTT CGG GAG GGT CGG GGG TAG CTC TTT TGG TAG AGG TTT CGG TTT CCC GTC | |
| Val Ser Asn Lys Ala Leu Pro Ala Pro Ile Glu Lys Thr Ile Ser Lys Ala Lys Gly Gln | |
| 741 745 hFC DOMAIN 755 760 | |
| CCC CGA GAA CCA CAG GTG TAC ACC CTG CCC CCA TCC CGG GAG GAG ATG ACC AAG AAC CAG | |
| GGG GCT CTT GGT GTC CAC ATG TGG GAC GGG GGT AGG GCC CTC CTC TAC TGG TTC TTG GTC | |
| Pro Arg Glu Pro Gln Val Tyr Thr Leu Pro Pro Ser Arg Glu Glu Met Thr Lys Asn Gln | |
| 761 765 hFC DOMAIN 775 780 | |
| GTC AGC CTG ACC TGC CTG GTC AAA GGC TTC TAT CCC AGC GAC ATC GCC GTG GAG TGG GAG | |
| CAG TCG GAC TGG ACG GAC CAG TTT CCG AAG ATA GGG TCG CTG TAG CGG CAC CTC ACC CTC | |
| Val Ser Leu Thr Cys Leu Val Lys Gly Phe Tyr Pro Ser Asp Ile Ala Val Glu Trp Glu | |
| 781 785 hFC DOMAIN 795 800 | |
| AGC AAT GGG CAG CCG GAG AAC AAC TAC AAG ACC ACG CCT CCC GTG CTG GAC TCC GAC GGC | |
| TCG TTA CCC GTC GGC CTC TTG TTG ATG TTC TGG TGC GGA GGG CAC GAC CTG AGG CTG CCG | |
| Ser Asn Gly Gln Pro Glu Asn Asn Tyr Lys Thr Thr Pro Pro Val Leu Asp Ser Asp Gly | |
| 801 805 hFC DOMAIN 815 820 | |
| TCC TTC TTC CTC TAC AGC AAG CTC ACC GTG GAC AAG AGC AGG TGG CAG CAG GGG AAC GTC | |
| AGG AAG AAG GAG ATG TCG TTC GAG TGG CAC CTG TTC TCG TCC ACC GTC GTC CCC TTG CAG | |
| Ser Phe Phe Leu Tyr Ser Lys Leu Thr Val Asp Lys Ser Arg Trp Gln Gln Gly Asn Val | |
| 821 825 hFC DOMAIN 835 840 | |
| TTC TCA TGC TCC GTG ATG CAT GAG GCT CTG CAC AAC CAC TAC ACG CAG AAG AGC CTC TCC | |
| AAG AGT ACG AGG CAC TAC GTA CTC CGA GAC GTG TTG GTG ATG TGC GTC TTC TCG GAG AGG | |
| Phe Ser Cys Ser Val Met His Glu Ala Leu His Asn His Tyr Thr Gln Lys Ser Leu Ser | |
| 841 845 hFC DOMAIN 855 860 | |
| CTG TCT CCG GGT AAA TGA | |
| GAC AGA GGC CCA TTT ACT | |
| Leu Ser Pro Gly Lys *** | |
| 861 865 866 | |
| TOY3 | |
| ATG CTA CTA GTA AAT CAG TCA CAC CAA GGC TTC AAT AAG GAA CAC ACA AGC AAG ATG GTA | (SEQ ID NO: 3) |
| TAC GAT GAT CAT TTA GTC AGT GTG GTT CCG AAG TTA TTC CTT GTG TGT TCG TTC TAC CAT | |
| Met Leu Leu Val Asn Gln Ser His Gln Gly Phe Asn Lys Glu His Thr Ser Lys Met Val | (SEQ ID NO: 4) |
| 1 5 gp67 secretion signal sequence 15 20 | |
| AGC GCT ATT GTT TTA TAT GTG CTT TTG GCG GCG GCG GCG CAT TCT GCC TTT GCG GCG GAT | |
| TCG CGA TAA CAA AAT ATA CAC GAA AAC CGC CGC CGC CGC GTA AGA CGG AAA CGC CGC CTA | |
| Ser Ala Ile Val Leu Tyr Val Leu Leu Ala Ala Ala Ala His Ser Ala Phe Ala Ala Asp | |
| 21 25 gp67 secretion signal sequence 35 38 40 | |
| CCC GAG CCC TGC GTG GAG GTG GTT CCT AAT ATT ACT TAT CAA TGC ATG GAG CTG AAT TTC | |
| GGG CTC GGG ACG CAC CTC CAC CAA GGA TTA TAA TGA ATA GTT ACG TAC CTC GAC TTA AAG | |
| Pro Glu Pro Cys Val Glu Val Val Pro Asn Ile Thr Tyr Gln Cys Met Glu Leu Asn Phe | |
| 41 42 45 LRRNT 55 60 | |
| TAC AAA ATC CCC GAC AAC CTC CCC TTC TCA ACC AAG AAC CTG GAC CTG AGC TTT AAT CCC | |
| ATG TTT TAG GGG CTG TTG GAG GGG AAG AGT TGG TTC TTG GAC CTG GAC TCG AAA TTA GGG | |
| Tyr Lys Ile Pro Asp Asn Leu Pro Phe Ser Thr Lys Asn Leu Asp Leu Ser Phe Asn Pro | |
| 61 LRRNT 66 67 LRR1 80 | |
| CTG AGG CAT TTA GGC AGC TAT AGC TTC TTC AGT TTC CCA GAA CTG CAG GTG CTG GAT TTA | |
| GAC TCC GTA AAT CCG TCG ATA TCG AAG AAG TCA AAG GGT CTT GAC GTC CAC GAC CTA AAT | |
| Leu Arg His Leu Gly Ser Tyr Ser Phe Phe Ser Phe Pro Glu Leu Gln Val Leu Asp Leu | |
| 81 LRR1 90 91 LRR2 100 | |
| TCC AGG TGT GAA ATC CAG ACA ATT GAA GAT GGG GCA TAT CAG AGC CTA AGC CAC CTC TCT | |
| AGG TCC ACA CTT TAG GTC TGT TAA CTT CTA CCC CGT ATA GTC TCG GAT TCG GTG GAG AGA | |
| Ser Arg Cys Glu Ile Gln Thr Ile Glu Asp Gly Ala Tyr Gln Ser Leu Ser His Leu Ser | |
| 101 LRR2 110 113 114 LRR3 120 | |
| ACC TTA ATA TTG ACA GGA AAC CCC ATC CAG AGT TTA GCC CTG GGA GCC TTT TCT GGA CTA | |
| TGG AAT TAT AAC TGT CCT TTG GGG TAG GTC TCA AAT CGG GAC CCT CGG AAA AGA CCT GAT | |
| Thr Leu Ile Leu Thr Gly Asn Pro Ile Gln Ser Leu Ala Leu Gly Ala Phe Ser Gly Leu | |
| 121 125 LRR3 138 139 140 | |
| TCA AGT TTA CAG AAG CTG GTG GCT GTG GAG ACA AAT CTA GCA TCT CTA GAG AAC TTC CCC | |
| AGT TCA AAT GTC TTC GAC CAC CGA CAC CTC TGT TTA GAT CGT AGA GAT CTC TTG AAG GGG | |
| Ser Ser Leu Gln Lys Leu Val Ala Val Glu Thr Asn Leu Ala Ser Leu Glu Asn Phe Pro | |
| 141 145 LRR4 155 160 | |
| ATT GGA CAT CTC AAA ACT TTG AAA GAA CTT AAT GTG GCT CAC AAT CTT ATC CAA TCT TTC | |
| TAA CCT GTA GAG TTT TGA AAC TTT CTT GAA TTA CAC CGA GTG TTA GAA TAG GTT AGA AAG | |
| Ile Gly His Leu Lys Thr Leu Lys Glu Leu Asn Val Ala His Asn Leu Ile Gln Ser Phe | |
| 161 162 163 LRR5 175 180 | |
| AAA TTA CCT GAG TAT TTT TCT AAT CTG ACC AAT CTA GAG CAC TTG GAC CTT TCC AGC AAC | |
| TTT AAT GGA CTC ATA AAA AGA TTA GAC TGG TTA GAT CTC GTG AAC CTG GAA AGG TCG TTG | |
| Lys Leu Pro Glu Tyr Phe Ser Asn Leu Thr Asn Leu Glu His Leu Asp Leu Ser Ser Asn | |
| 181 LRR5 187 188 LRR6 200 | |
| AAG ATT CAA AGT ATT TAT TGC ACA GAC TTG CGG GTT CTA CAT CAA ATG CCC CTA CTC AAT | |
| TTC TAA GTT TCA TAA ATA ACG TGT CTG AAC GCC CAA GAT GTA GTT TAC GGG GAT GAG TTA | |
| Lys Ile Gln Ser Ile Tyr Cys Thr Asp Leu Arg Val Leu His Gln Met Pro Leu Leu Asn | |
| 201 205 LRR6 217 218 220 | |
| CTC TCT TTA GAC CTG TCC CTG AAC CCT ATG AAC TTT ATC CAA CCA GGT GCA TTT AAA GAA | |
| GAG AGA AAT CTG GAC AGG GAC TTG GGA TAC TTG AAA TAG GTT GGT CCA CGT AAA TTT CTT | |
| Leu Ser Leu Asp Leu Ser Leu Asn Pro Met Asn Phe Ile Gln Pro Gly Ala Phe Lys Glu | |
| 221 225 LRR7 235 239 240 | |
| ATT AGG CTC AAA GAG CTC GCC CTG GAC ACC AAC CAG CTG AAG TCT GTT CCT GAT GGG ATT | |
| TAA TCC GAG TTT CTC GAG CGG GAC CTG TGG TTG GTC GAC TTC AGA CAA GGA CTA CCC TAA | |
| Ile Arg Leu Lys Glu Leu Ala Leu Asp Thr Asn Gln Leu Lys Ser Val Pro Asp Gly Ile | |
| 241 242 243 VLRB.61_hybrid1 260 | |
| TTT GAT CGC CTG ACC AGC TTG CAG AAA ATT TGG CTT CAT ACA AAC CCT TGG GAC TGC AGT | |
| AAA CTA GCG GAC TGG TCG AAC GTC TTT TAA ACC GAA GTA TGT TTG GGA ACC CTG ACG TCA | |
| Phe Asp Arg Leu Thr Ser Leu Gln Lys Ile Trp Leu His Thr Asn Pro Trp Asp Cys Ser | |
| 261 VLRB.61_hybrid1 280 | |
| TGT CCC AGG ATT GAC TAT CTC AGT CGC TGG TTA AAT AAA AAC TCA CAG AAG GAA CAG GGC | |
| ACA GGG TCC TAA CTG ATA GAG TCA GCG ACC AAT TTA TTT TTG AGT GTC TTC CTT GTC CCG | |
| Cys Pro Arg Ile Asp Tyr Leu Ser Arg Trp Leu Asn Lys Asn Ser Gln Lys Glu Gln Gly | |
| 281 VLRB.61_hybrid1 300 | |
| AGT GCT AAA TGT TCC GGG TCC GGC AAG CCC GTC CGA AGT ATC ATC TGC CCT ACT CTC GAG | |
| TCA CGA TTT ACA AGG CCC AGG CCG TTC GGG CAG GCT TCA TAG TAG ACG GGA TGA GAG CTC | |
| Ser Ala Lys Cys Ser Gly Ser Gly Lys Pro Val Arg Ser Ile Ile Cys Pro Thr Leu Glu | |
| 301 VLRB.61_hybrid1 318 319 hFC | |
| GAC AAA ACT CAC ACA TGC CCA CCG TGC CCA GCA CCT GAA CTC CTG GGG GGA CCG TCA GTC | |
| CTG TTT TGA GTG TGT ACG GGT GGC ACG GGT CGT GGA CTT GAG GAC CCC CCT GGC AGT CAG | |
| Asp Lys Thr His Thr Cys Pro Pro Cys Pro Ala Pro Glu Leu Leu Gly Gly Pro Ser Val | |
| DOMAIN 325 hFC DOMAIN 335 340 | |
| TTC CTC TTC CCC CCA AAA CCC AAG GAC ACC CTC ATG ATC TCC CGG ACC CCT GAG GTC ACA | |
| AAG GAG AAC GGG GGT TTT GGG TTC CTG TGG GAG TAC TAG AGG GCC TGG GGA CTC CAG TGT | |
| Phe Leu Phe Pro Pro Lys Pro Lys Asp Thr Leu Met Ile Ser Arg Thr Pro Glu Val Thr | |
| 341 345 hFC DOMAIN 355 360 | |
| TGC GTG GTG GTG GAC GTG AGC CAC GAA GAC CCT GAG GTC AAG TTC AAC TGG TAC GTG GAC | |
| ACG CAC CAC CAC CTG CAC TCG GTG CTT CTG GGA CTC CAG TTC AAG TTG ACC ATG CAC CTG | |
| Cys Val Val Val Asp Val Ser His Glu Asp Pro Glu Val Lys Phe Asn Trp Tyr Val Asp | |
| 361 365 hFC DOMAIN 375 380 | |
| GGC GTG GAG GTG CAT AAT GCC AAG ACA AAG CCG CGG GAG GAG CAG TAC AAC AGC ACG TAC | |
| CCG CAC CTC CAC GTA TTA CGG TTC TGT TTC GGC GCC CTC CTC GTC ATG TTG TCG TGC ATG | |
| Gly Val Glu Val His Asn Ala Lys Thr Lys Pro Arg Glu Glu Gln Tyr Asn Ser Thr Tyr | |
| 381 385 hFC DOMAIN 395 400 | |
| CGT GTG GTC AGC GTC CTC ACC GTC CTG CAC CAG GAC TGG CTG AAT GGC AAG GAG TAC AAG | |
| GCA CAC CAG TCG CAG GAG TGG CAG GAC GTG GTC CTG ACC GAC TTA CCG TTC CTC ATG TTC | |
| Arg Val Val Ser Val Leu Thr Val Leu His Gln Asp Trp Leu Asn Gly Lys Glu Tyr Lys | |
| 401 405 hFC DOMAIN 415 420 | |
| TGC AAG GTC TCC AAC AAA GCC CTC CCA GCC CCC ATC GAG AAA ACC ATC TCC AAA GCC AAA | |
| ACG TTC CAG AGG TTG TTT CGG GAG GGT CGG GGG TAG CTC TTT TGG TAG AGG TTT CGG TTT | |
| Cys Lys Val Ser Asn Lys Ala Leu Pro Ala Pro Ile Glu Lys Thr Ile Ser Lys Ala Lys | |
| 421 425 hFC DOMAIN 435 440 | |
| GGG CAG CCC CGA GAA CCA CAG GTG TAC ACC CTG CCC CCA TCC CGG GAG GAG ATG ACC AAG | |
| CCC GTC GGG GCT CTT GGT GTC CAC ATG TGG GAC GGG GGT AGG GCC CTC CTC TAC TGG TTC | |
| Gly Gln Pro Arg Glu Pro Gln Val Tyr Thr Leu Pro Pro Ser Arg Glu Glu Met Thr Lys | |
| 441 445 hFC DOMAIN 455 460 | |
| AAC CAG GTC AGC CTG ACC TGC CTG GTC AAA GGC TTC TAT CCC AGC GAC ATC GCC GTG GAG | |
| TTG GTC CAG TCG GAC TGG ACG GAC CAG TTT CCG AAG ATA GGG TCG CTG TAG CGG CAC CTC | |
| Asn Gln Val Ser Leu Thr Cys Leu Val Lys Gly Phe Tyr Pro Ser Asp Ile Ala Val Glu | |
| 461 465 hFC DOMAIN 475 480 | |
| TGG GAG AGC AAT GGG CAG CCG GAG AAC AAC TAC AAG ACC ACG CCT CCC GTG CTG GAC TCC | |
| ACC CTC TCG TTA CCC GTC GGC CTC TTG TTG ATG TTC TGG TGC GGA GGG CAC GAC CTG AGG | |
| Trp Glu Ser Asn Gly Gln Pro Glu Asn Asn Tyr Lys Thr Thr Pro Pro Val Leu Asp Ser | |
| 481 485 hFC DOMAIN 495 500 | |
| GAC GGC TCC TTC TTC CTC TAC AGC AAG CTC ACC GTG GAC AAG AGC AGG TGG CAG CAG GGG | |
| CTG CCG AGG AAG AAG GAG ATG TCG TTC GAG TGG CAC CTG TTC TCG TCC ACC GTC GTC CCC | |
| Asp Gly Ser Phe Phe Leu Tyr Ser Lys Leu Thr Val Asp Lys Ser Arg Trp Gln Gln Gly | |
| 501 505 hFC DOMAIN 515 520 | |
| AAC GTC TTC TCA TGC TCC GTG ATG CAT GAG GCT CTG CAC AAC CAC TAC ACG CAG AAG AGC | |
| TTG CAG AAG AGT ACG AGG CAC TAC GTA CTC CGA GAC GTG TTG GTG ATG TGC GTC TTC TCG | |
| Asn Val Phe Ser Cys Ser Val Met His Glu Ala Leu His Asn His Tyr Thr Gln Lys Ser | |
| 521 525 hFC DOMAIN 535 540 | |
| CTC TCC CTG TCT CCG GGT AAA TGA | |
| GAG AGG GAC AGA GGC CCA TTT ACT | |
| Leu Ser Leu Ser Pro Gly Lys *** | |
| 541 545 548 | |
| TOY8 | |
| ATG CTA CTA GTA AAT CAG TCA CAC CAA GGC TTC AAT AAG GAA CAC ACA AGC AAG ATG GTA | (SEQ ID NO: 5) |
| TAC GAT GAT CAT TTA GTC AGT GTG GTT CCG AAG TTA TTC CTT GTG TGT TCG TTC TAC CAT | |
| Met Leu Leu Val Asn Gln Ser His Gln Gly Phe Asn Lys Glu His Thr Ser Lys Met Val | (SEQ ID NO: 6) |
| 1 5 gp67 secretion signal sequence 15 20 | |
| AGC GCT ATT GTT TTA TAT GTG CTT TTG GCG GCG GCG GCG CAT TCT GCC TTT GCG GCG GAT | |
| TCG CGA TAA CAA AAT ATA CAC GAA AAC CGC CGC CGC CGC GTA AGA CGG AAA CGC CGC CTA | |
| Ser Ala Ile Val Leu Tyr Val Leu Leu Ala Ala Ala Ala His Ser Ala Phe Ala Ala Asp | |
| 21 25 gp67 secretion signal sequence 35 38 40 | |
| CCC GAG CCC TGC GTG GAG GTG GTT CCT AAT ATT ACT TAT CAA TGC ATG GAG CTG AAT TTC | |
| GGG CTC GGG ACG CAC CTC CAC CAA GGA TTA TAA TGA ATA GTT ACG TAC CTC GAC TTA AAG | |
| Pro Glu Pro Cys Val Glu Val Val Pro Asn Ile Thr Tyr Gln Cys Met Glu Leu Asn Phe | |
| 41 42 45 LRRNT 55 60 | |
| TAC AAA ATC CCC GAC AAC CTC CCC TTC TCA ACC AAG AAC CTG GAC CTG AGC TTT AAT CCC | |
| ATG TTT TAG GGG CTG TTG GAG GGG AAG AGT TGG TTC TTG GAC CTG GAC TCG AAA TTA GGG | |
| Tyr Lys Ile Pro Asp Asn Leu Pro Phe Ser Thr Lys Asn Leu Asp Leu Ser Phe Asn Pro | |
| 61 LRRNT 66 67 LRR1 80 | |
| CTG AGG CAT TTA GGC AGC TAT AGC TTC TTC AGT TTC CCA GAA CTG CAG GTG CTG GAT TTA | |
| GAC TCC GTA AAT CCG TCG ATA TCG AAG AAG TCA AAG GGT CTT GAC GTC CAC GAC CTA AAT | |
| Leu Arg His Leu Gly Ser Tyr Ser Phe Phe Ser Phe Pro Glu Leu Gln Val Leu Asp Leu | |
| 81 LRR1 90 91 LRR2 100 | |
| TCC AGG TGT GAA ATC CAG ACA ATT GAA GAT GGG GCA TAT CAG AGC CTA AGC CAC CTC TCT | |
| AGG TCC ACA CTT TAG GTC TGT TAA CTT CTA CCC CGT ATA GTC TCG GAT TCG GTG GAG AGA | |
| Ser Arg Cys Glu Ile Gln Thr Ile Glu Asp Gly Ala Tyr Gln Ser Leu Ser His Leu Ser | |
| 101 LRR2 110 113 114 LRR3 120 | |
| ACC TTA ATA TTG ACA GGA AAC CCC ATC CAG AGT TTA GCC CTG GGA GCC TTT TCT GGA CTA | |
| TGG AAT TAT AAC TGT CCT TTG GGG TAG GTC TCA AAT CGG GAC CCT CGG AAA AGA CCT GAT | |
| Thr Leu Ile Leu Thr Gly Asn Pro Ile Gln Ser Leu Ala Leu Gly Ala Phe Ser Gly Leu | |
| 121 125 LRR3 138 139 140 | |
| TCA AGT TTA CAG AAG CTG GTG GCT GTG GAG ACA AAT CTA GCA TCT CTA GAG AAC TTC CCC | |
| AGT TCA AAT GTC TTC GAC CAC CGA CAC CTC TGT TTA GAT CGT AGA GAT CTC TTG AAG GGG | |
| Ser Ser Leu Gln Lys Leu Val Ala Val Glu Thr Asn Leu Ala Ser Leu Glu Asn Phe Pro | |
| 141 145 LRR4 155 160 | |
| ATT GGA CAT CTC AAA ACT TTG AAA GAA CTT AAT GTG GCT CAC AAT CTT ATC CAA TCT TTC | |
| TAA CCT GTA GAG TTT TGA AAC TTT CTT GAA TTA CAC CGA GTG TTA GAA TAG GTT AGA AAG | |
| Ile Gly His Leu Lys Thr Leu Lys Glu Leu Asn Val Ala His Asn Leu Ile Gln Ser Phe | |
| 161 162 163 LRR5 175 180 | |
| AAA TTA CCT GAG TAT TTT TCT AAT CTG ACC AAT CTA GAG CAC TTG GAC CTT TCC AGC AAC | |
| TTT AAT GGA CTC ATA AAA AGA TTA GAC TGG TTA GAT CTC GTG AAC CTG GAA AGG TCG TTG | |
| Lys Leu Pro Glu Tyr Phe Ser Asn Leu Thr Asn Leu Glu His Leu Asp Leu Ser Ser Asn | |
| 181 LRR5 187 188 LRR6 200 | |
| AAG ATT CAA AGT ATT TAT TGC ACA GAC TTG CGG GTT CTA CAT CAA ATG CCC CTA CTC AAT | |
| TTC TAA GTT TCA TAA ATA ACG TGT CTG AAC GCC CAA GAT GTA GTT TAC GGG GAT GAG TTA | |
| Lys Ile Gln Ser Ile Tyr Cys Thr Asp Leu Arg Val Leu His Gln Met Pro Leu Leu Asn | |
| 201 205 LRR6 217 218 220 | |
| CTC TCT TTA GAC CTG TCC CTG AAC CCT ATG AAC TTT ATC CAA CCA GGT GCA TTT AAA GAA | |
| GAG AGA AAT CTG GAC AGG GAC TTG GGA TAC TTG AAA TAG GTT GGT CCA CGT AAA TTT CTT | |
| Leu Ser Leu Asp Leu Ser Leu Asn Pro Met Asn Phe Ile Gln Pro Gly Ala Phe Lys Glu | |
| 221 225 LRR7 235 239 240 | |
| ATT AGG CTT CAT AAG CTG ACT TTA AGA AAT AAT TTT GAT AGT TTA AAT GTA ATG AAA ACT | |
| TAA TCC GAA GTA TTC GAC TGA AAT TCT TTA TTA AAA CTA TCA AAT TTA CAT TAC TTT TGA | |
| Ile Arg Leu His Lys Leu Thr Leu Arg Asn Asn Phe Asp Ser Leu Asn Val Met Lys Thr | |
| 241 245 LRR8 255 260 | |
| TGT ATT CAA GGT CTG GCT GGT TTA GAA GTC CAT CGT TTG GTT CTG GGA GAA TTT AGA AAT | |
| ACA TAA GTT CCA GAC CGA CCA AAT CTT CAG GTA GCA AAC CAA GAC CCT CTT AAA TCT TTA | |
| Cys Ile Gln Gly Leu Ala Gly Leu Glu Val His Arg Leu Val Leu Gly Glu Phe Arg Asn | |
| 261 LRR8 266 267 LRR9 280 | |
| GAA GGA AAC TTG GAA AAG TTT GAC AAA TCT GCT CTA GAG GGC CTG TGC AAT TTG ACC ATT | |
| CTT CCT TTG AAC CTT TTC AAA CTG TTT AGA CGA GAT CTC CCG GAC ACG TTA AAC TGG TAA | |
| Glu Gly Asn Leu Glu Lys Phe Asp Lys Ser Ala Leu Glu Gly Leu Cys Asn Leu Thr Ile | |
| 281 285 LRR9 296 297 LRR10 300 | |
| GAA GAA TTC CGA TTA GCA TAC TTA GAC TAC TAC CTC GAT GAT ATT ATT GAC TTA TTT AAT | |
| CTT CTT AAG GCT AAT CGT ATG AAT CTG ATG ATG GAG CTA CTA TAA TAA CTG AAT AAA TTA | |
| Glu Glu Phe Arg Leu Ala Tyr Leu Asp Tyr Tyr Leu Asp Asp Ile Ile Asp Leu Phe Asn | |
| 301 305 LRR10 315 320 | |
| TGT TTG ACA AAT GTT TCT TCA TTT TCC CTG GTG AGT GTG ACT ATT GAA AGG GTA AAA GAC | |
| ACA AAC TGT TTA CAA AGA AGT AAA AGG GAC CAC TCA CAC TGA TAA CTT TCC CAT TTT CTG | |
| Cys Leu Thr Asn Val Ser Ser Phe Ser Leu Val Ser Val Thr Ile Glu Arg Val Lys Asp | |
| 321 325 LRR11 335 340 | |
| TTT TCT TAT AAT TTC GGA TGG CAA CAT TTA GAA TTA GTT AAC TGT AAA TTT GGA CAG TTT | |
| AAA AGA ATA TTA AAG CCT ACC GTT GTA AAT CTT AAT CAA TTG ACA TTT AAA CCT GTC AAA | |
| Phe Ser Tyr Asn Phe Gly Trp Gln His Leu Glu Leu Val Asn Cys Lys Phe Gly Gln Phe | |
| 341 343 344 LRR12 355 360 | |
| CCC ACA TTG AAA CTC AAA TCT CTC AAA AGG CTT ACT TTC ACT TCC AAC AAA GGT GGG AAT | |
| GGG TGT AAC TTT GAG TTT AGA GAG TTT TCC GAA TGA AAG TGA AGG TTG TTT CCA CCC TTA | |
| Pro Thr Leu Lys Leu Lys Ser Leu Lys Arg Leu Thr Phe Thr Ser Asn Lys Gly Gly Asn | |
| 361 363 364 LRR13 375 380 | |
| GCT TTT TCA GAA GTT GAT CTA CCA AGC CTT GAG TTT CTA GAT CTC AGT AGA AAT GGC TTG | |
| CGA AAA AGT CTT CAA CTA GAT GGT TCG GAA CTC AAA GAT CTA GAG TCA TCT TTA CCG AAC | |
| Ala Phe Ser Glu Val Asp Leu Pro Ser Leu Glu Phe Leu Asp Leu Ser Arg Asn Gly Leu | |
| 381 LRR13 385 386 LRR14 395 400 | |
| AGT TTC AAA GGT TGC TGT TCT CAA AGT GAT TTT GGG ACA ACC AGC CTA AAG TAT TTA GAT | |
| TCA AAG TTT CCA ACG ACA AGA GTT TCA CTA AAA CCC TGT TGG TCG GAT TTC ATA AAT CTA | |
| Ser Phe Lys Gly Cys Cys Ser Gln Ser Asp Phe Gly Thr Thr Ser Leu Lys Tyr Leu Asp | |
| 401 LRR14 411 412 LRR15 420 | |
| CTG AGC TTC AAT GGT GTT ATT ACC ATG AGT TCA AAC TTC TTG GGC TTA GAA CAA CTA GAA | |
| GAC TCG AAG TTA CCA CAA TAA TGG TAC TCA AGT TTG AAG AAC CCG AAT CTT GTT GAT CTT | |
| Leu Ser Phe Asn Gly Val Ile Thr Met Ser Ser Asn Phe Leu Gly Leu Glu Gln Leu Glu | |
| 421 LRR15 434 435 LRR16 440 | |
| CAT CTG GAT TTC CAG CAT TCC AAT TTG AAA CAA ATG AGT GAG TTT TCA GTA TTC CTA TCA | |
| GTA GAC CTA AAG GTC GTA AGG TTA AAC TTT GTT TAC TCA CTC AAA AGT CAT AAG GAT AGT | |
| His Leu Asp Phe Gln His Ser Asn Leu Lys Gln Met Ser Glu Phe Ser Val Phe Leu Ser | |
| 441 LRR16 459 460 | |
| CTC AGA AAC CTC ATT TAC CTT GAC ATT TCT CAT ACT CAC ACC AGA GTT GCT TTC AAT GGC | |
| GAG TCT TTG GAG TAA ATG GAA CTG TAA AGA GTA TGA GTG TGG TCT CAA CGA AAG TTA CCG | |
| Leu Arg Asn Leu Ile Tyr Leu Asp Ile Ser His Thr His Thr Arg Val Ala Phe Asn Gly | |
| 461 465 LRR17 475 480 | |
| ATC TTC AAT GGC TTG TCC AGT CTC GAA GTC TTG AAA ATG GCT GGC AAT TCT TTC CAG GAA | |
| TAG AAG TTA CCG AAC AGG TCA GAG CTT CAG AAC TTT TAC CGA CCG TTA AGA AAG GTC CTT | |
| Ile Phe Asn Gly Leu Ser Ser Leu Glu Val Leu Lys Met Ala Gly Asn Ser Phe Gln Glu | |
| 481 483 484 LRR18 495 500 | |
| AAC TTC CTT CCA GAT ATC TTC ACA GAG CTG AGA AAC TTG ACC TTC CTG GAC CTC TCT CAG | |
| TTG AAG GAA GGT CTA TAG AAG TGT CTC GAC TCT TTG AAC TGG AAG GAC CTG GAG AGA GTC | |
| Asn Phe Leu Pro Asp Ile Phe Thr Glu Leu Arg Asn Leu Thr Phe Leu Asp Leu Ser Gln | |
| 501 LRR18 508 509 LRR19 520 | |
| TGT CAA CTG GAG CAG TTG TCT CCA ACA GCA TTT AAC TCA CTC TCC AGT CTT CAG GTA CTA | |
| ACA GTT GAC CTC GTC AAC AGA GGT TGT CGT AAA TTG AGT GAG AGG TCA GAA GTC CAT GAT | |
| Cys Gln Leu Glu Gln Leu Ser Pro Thr Ala Phe Asn Ser Leu Ser Ser Leu Gln Val Leu | |
| 521 LRR19 532 533 LRR20 540 | |
| AAT ATG GCT AGT AAC CAG CTG AAG TCT GTT CCT GAT GGG ATT TTT GAT CGC CTG ACC AGC | |
| TTA TAC CGA TCA TTG GTC GAC TTC AGA CAA GGA CTA CCC TAA AAA CTA GCG GAC TGG TCG | |
| Asn Met Ala Ser Asn Gln Leu Lys Ser Val Pro Asp Gly Ile Phe Asp Arg Leu Thr Ser | |
| 541 542 545 VLRB.61_hybrid2 560 | |
| TTG CAG AAA ATT TGG CTT CAT ACA AAC CCT TGG GAC TGC AGT TGT CCC AGG ATT GAC TAT | |
| AAC GTC TTT TAA ACC GAA GTA TGT TTG GGA ACC CTG ACG TCA ACA GGG TCC TAA CTG ATA | |
| Leu Gln Lys Ile Trp Leu His Thr Asn Pro Trp Asp Cys Ser Cys Pro Arg Ile Asp Tyr | |
| 561 VLRB.61_hybrid2 580 | |
| CTC AGT CGC TGG TTA AAT AAA AAC TCA CAG AAG GAA CAG GGC AGT GCT AAA TGT TCC GGG | |
| GAG TCA GCG ACC AAT TTA TTT TTG AGT GTC TTC CTT GTC CCG TCA CGA TTT ACA AGG CCC | |
| Leu Ser Arg Trp Leu Asn Lys Asn Ser Gln Lys Glu Gln Gly Ser Ala Lys Cys Ser Gly | |
| 581 VLRB.61_hybrid2 600 | |
| TCC GGC AAG CCC GTC CGA AGT ATC ATC TGC CCT ACT CTC GAG GAC AAA ACT CAC ACA TGC | |
| AGG CCG TTC GGG CAG GCT TCA TAG TAG ACG GGA TGA GAG CTC CTG TTT TGA GTG TGT ACG | |
| Ser Gly Lys Pro Val Arg Ser Ile Ile Cys Pro Thr Leu Glu Asp Lys Thr His Thr Cys | |
| 601 VLRB.61_hybrid2 612 613 hFC DOMAIN 620 | |
| CCA CCG TGC CCA GCA CCT GAA CTC CTG GGG GGA CCG TCA GTC TTC CTC TTC CCC CCA AAA | |
| GGT GGC ACG GGT CGT GGA CTT GAG GAC CCC CCT GGC AGT CAG AAG GAG AAG GGG GGT TTT | |
| Pro Pro Cys Pro Ala Pro Glu Leu Leu Gly Gly Pro Ser Val Phe Leu Phe Pro Pro Lys | |
| 621 625 hFC DOMAIN 635 640 | |
| CCC AAG GAC ACC CTC ATG ATC TCC CGG ACC CCT GAG GTC ACA TGC GTG GTG GTG GAC GTG | |
| GGG TTC CTG TGG GAG TAC TAG AGG GCC TGG GGA CTC CAG TGT ACG CAC CAC CAC CTG CAC | |
| Pro Lys Asp Thr Leu Met Ile Ser Arg Thr Pro Glu Val Thr Cys Val Val Val Asp Val | |
| 641 645 hFC DOMAIN 655 660 | |
| AGC CAC GAA GAC CCT GAG GTC AAG TTC AAC TGG TAC GTG GAC GGC GTG GAG GTG CAT AAT | |
| TCG GTG CTT CTG GGA CTC CAG TTC AAG TTG ACC ATG CAC CTG CCG CAC CTC CAC GTA TTA | |
| Ser His Glu Asp Pro Glu Val Lys Phe Asn Trp Tyr Val Asp Gly Val Glu Val His Asn | |
| 661 665 hFC DOMAIN 675 680 | |
| GCC AAG ACA AAG CCG CGG GAG GAG CAG TAC AAC AGC ACG TAC CGT GTG GTC AGC GTC CTC | |
| CGG TTC TGT TTC GGC GCC CTC CTC GTC ATG TTG TCG TGC ATG GCA CAC CAG TCG CAG GAG | |
| Ala Lys Thr Lys Pro Arg Glu Glu Gln Tyr Asn Ser Thr Tyr Arg Val Val Ser Val Leu | |
| 681 685 hFC DOMAIN 695 700 | |
| ACC GTC CTG CAC CAG GAC TGG CTG AAT GGC AAG GAG TAC AAG TGC AAG GTC TCC AAC AAA | |
| TGG CAG GAC GTG GTC CTG ACC GAC TTA CCG TTC CTC ATG TTC ACG TTC CAG AGG TTG TTT | |
| Thr Val Leu His Gln Asp Trp Leu Asn Gly Lys Glu Tyr Lys Cys Lys Val Ser Asn Lys | |
| 701 705 hFC DOMAIN 715 720 | |
| GCC CTC CCA GCC CCC ATC GAG AAA ACC ATC TCC AAA GCC AAA GGG CAG CCC CGA GAA CCA | |
| CGG GAG GGT CGG GGG TAG CTC TTT TGG TAG AGG TTT CGG TTT CCC GTC GGG GCT CTT GGT | |
| Ala Leu Pro Ala Pro Ile Glu Lys Thr Ile Ser Lys Ala Lys Gly Gln Pro Arg Glu Pro | |
| 721 725 hFC DOMAIN 735 740 | |
| CAG GTG TAC ACC CTG CCC CCA TCC CGG GAG GAG ATG ACC AAG AAC CAG GTC AGC CTG ACC | |
| GTC CAC ATG TGG GAC GGG GGT AGG GCC CTC CTC TAC TGG TTC TTG GTC CAG TCG GAC TGG | |
| Gln Val Tyr Thr Leu Pro Pro Ser Arg Glu Glu Met Thr Lys Asn Gln Val Ser Leu Thr | |
| 741 745 hFC DOMAIN 755 760 | |
| TGC CTG GTC AAA GGC TTC TAT CCC AGC GAC ATC GCC GTG GAG TGG GAG AGC AAT GGG CAG | |
| ACG GAC CAG TTT CCG AAG ATA GGG TCG CTG TAG CGG CAC CTC ACC CTC TCG TTA CCC GTC | |
| Cys Leu Val Lys Gly Phe Tyr Pro Ser Asp Ile Ala Val Glu Trp Glu Ser Asn Gly Gln | |
| 761 765 hFC DOMAIN 775 780 | |
| CCG GAG AAC AAC TAC AAG ACC ACG CCT CCC GTG CTG GAC TCC GAC GGC TCC TTC TTC CTC | |
| GGC CTC TTG TTG ATG TTC TGG TGC GGA GGG CAC GAC CTG AGG CTG CCG AGG AAG AAG GAG | |
| Pro Glu Asn Asn Tyr Lys Thr Thr Pro Pro Val Leu Asp Ser Asp Gly Ser Phe Phe Leu | |
| 781 785 hFC DOMAIN 795 800 | |
| TAC AGC AAG CTC ACC GTG GAC AAG AGC AGG TGG CAG CAG GGG AAC GTC TTC TCA TGC TCC | |
| ATG TCG TTC GAG TGG CAC CTG TTC TCG TCC ACC GTC GTC CCC TTG CAG AAG AGT ACG AGG | |
| Tyr Ser Lys Leu Thr Val Asp Lys Ser Arg Trp Gln Gln Gly Asn Val Phe Ser Cys Ser | |
| 801 805 hFC DOMAIN 815 820 | |
| GTG ATG CAT GAG GCT CTG CAC AAC CAC TAC ACG CAG AAG AGC CTC TCC CTG TCT CCG GGT | |
| CAC TAC GTA CTC CGA GAC GTG TTG GTG ATG TGC GTC TTC TCG GAG AGG GAC AGA GGC CCA | |
| Val Met His Glu Ala Leu His Asn His Tyr Thr Gln Lys Ser Leu Ser Leu Ser Pro Gly | |
| 821 825 hFC DOMAIN 835 840 | |
| AAA TGA | |
| TTT ACT | |
| Lys *** | |
| 841 842 | |
| TOY9 | |
| ATG CTA CTA GTA AAT CAG TCA CAC CAA GGC TTC AAT AAG GAA CAC ACA AGC AAG ATG GTA | (SEQ ID NO: 7) |
| TAC GAT GAT CAT TTA GTC AGT GTG GTT CCG AAG TTA TTC CTT GTG TGT TCG TTC TAC CAT | (SEQ ID NO: 8) |
| Met Leu Leu Val Asn Gln Ser His Gln Gly Phe Asn Lys Glu His Thr Ser Lys Met Val | |
| 1 5 gp67 secretion signal sequence 15 20 | |
| AGC GCT ATT GTT TTA TAT GTG CTT TTG GCG GCG GCG GCG CAT TCT GCC TTT GCG GCG GAT | |
| TCG CGA TAA CAA AAT ATA CAC GAA AAC CGC CGC CGC CGC GTA AGA CGG AAA CGC CGC CTA | |
| Ser Ala Ile Val Leu Tyr Val Leu Leu Ala Ala Ala Ala His Ser Ala Phe Ala Ala Asp | |
| 21 25 gp67 secretion signal sequence 35 38 40 | |
| CCC GAG CCC TGC GTG GAG GTG GTT CCT AAT ATT ACT TAT CAA TGC ATG GAG CTG AAT TTC | |
| GGG CTC GGG ACG CAC CTC CAC CAA GGA TTA TAA TGA ATA GTT ACG TAC CTC GAC TTA AAG | |
| Pro Glu Pro Cys Val Glu Val Val Pro Asn Ile Thr Tyr Gln Cys Met Glu Leu Asn Phe | |
| 41 42 45 LRRNT 55 60 | |
| TAC AAA ATC CCC GAC AAC CTC CCC TTC TCA ACC AAG AAC CTG GAC CTG AGC TTT AAT CCC | |
| ATG TTT TAG GGG CTG TTG GAG GGG AAG AGT TGG TTC TTG GAC CTG GAC TCG AAA TTA GGG | |
| Tyr Lys Ile Pro Asp Asn Leu Pro Phe Ser Thr Lys Asn Leu Asp Leu Ser Phe Asn Pro | |
| 61 LRRNT 66 67 LRR1 80 | |
| CTG AGG CAT TTA GGC AGC TAT AGC TTC TTC AGT TTC CCA GAA CTG CAG GTG CTG GAT TTA | |
| GAC TCC GTA AAT CCG TCG ATA TCG AAG AAG TCA AAG GGT CTT GAC GTC CAC GAC CTA AAT | |
| Leu Arg His Leu Gly Ser Tyr Ser Phe Phe Ser Phe Pro Glu Leu Gln Val Leu Asp Leu | |
| 81 LRR1 90 91 LRR2 100 | |
| TCC AGG TGT GAA ATC CAG ACA ATT GAA GAT GGG GCA TAT CAG AGC CTA AGC CAC CTC TCT | |
| AGG TCC ACA CTT TAG GTC TGT TAA CTT CTA CCC CGT ATA GTC TCG GAT TCG GTG GAG AGA | |
| Ser Arg Cys Glu Ile Gln Thr Ile Glu Asp Gly Ala Tyr Gln Ser Leu Ser His Leu Ser | |
| 101 LRR2 110 113 114 LRR3 120 | |
| ACC TTA ATA TTG ACA GGA AAC CCC ATC CAG AGT TTA GCC CTG GGA GCC TTT TCT GGA CTA | |
| TGG AAT TAT AAC TGT CCT TTG GGG TAG GTC TCA AAT CGG GAC CCT CGG AAA AGA CCT GAT | |
| Thr Leu Ile Leu Thr Gly Asn Pro Ile Gln Ser Leu Ala Leu Gly Ala Phe Ser Gly Leu | |
| 121 125 LRR3 138 139 140 | |
| TCA AGT TTA CAG AAG CTG GTG GCT GTG GAG ACA AAT CTA GCA TCT CTA GAG AAC TTC CCC | |
| AGT TCA AAT GTC TTC GAC CAC CGA CAC CTC TGT TTA GAT CGT AGA GAT CTC TTG AAG GGG | |
| Ser Ser Leu Gln Lys Leu Val Ala Val Glu Thr Asn Leu Ala Ser Leu Glu Asn Phe Pro | |
| 141 145 LRR4 155 160 | |
| ATT GGA CAT CTC AAA ACT TTG AAA GAA CTT AAT GTG GCT CAC AAT CTT ATC CAA TCT TTC | |
| TAA CCT GTA GAG TTT TGA AAC TTT CTT GAA TTA CAC CGA GTG TTA GAA TAG GTT AGA AAG | |
| Ile Gly His Leu Lys Thr Leu Lys Glu Leu Asn Val Ala His Asn Leu Ile Gln Ser Phe | |
| 161 162 163 LRR5 175 180 | |
| AAA TTA CCT GAG TAT TTT TCT AAT CTG ACC AAT CTA GAG CAC TTG GAC CTT TCC AGC AAC | |
| TTT AAT GGA CTC ATA AAA AGA TTA GAC TGG TTA GAT CTC GTG AAC CTG GAA AGG TCG TTG | |
| Lys Leu Pro Glu Tyr Phe Ser Asn Leu Thr Asn Leu Glu His Leu Asp Leu Ser Ser Asn | |
| 181 LRR5 187 188 LRR6 200 | |
| AAG ATT CAA AGT ATT TAT TGC ACA GAC TTG CGG GTT CTA CAT CAA ATG CCC CTA CTC AAT | |
| TTC TAA GTT TCA TAA ATA ACG TGT CTG AAC GCC CAA GAT GTA GTT TAC GGG GAT GAG TTA | |
| Lys Ile Gln Ser Ile Tyr Cys Thr Asp Leu Arg Val Leu His Gln Met Pro Leu Leu Asn | |
| 201 205 LRR6 217 218 220 | |
| CTC TCT TTA GAC CTG TCC CTG AAC CCT ATG AAC TTT ATC CAA CCA GGT GCA TTT AAA GAA | |
| GAG AGA AAT CTG GAC AGG GAC TTG GGA TAC TTG AAA TAG GTT GGT CCA CGT AAA TTT CTT | |
| Leu Ser Leu Asp Leu Ser Leu Asn Pro Met Asn Phe Ile Gln Pro Gly Ala Phe Lys Glu | |
| 221 225 LRR7 235 239 240 | |
| ATT AGG CTT CAT AAG CTG ACT TTA AGA AAT AAT TTT GAT AGT TTA AAT GTA ATG AAA ACT | |
| TAA TCC GAA GTA TTC GAC TGA AAT TCT TTA TTA AAA CTA TCA AAT TTA CAT TAC TTT TGA | |
| Ile Arg Leu His Lys Leu Thr Leu Arg Asn Asn Phe Asp Ser Leu Asn Val Met Lys Thr | |
| 241 245 LRR8 255 260 | |
| TGT ATT CAA GGT CTG GCT GGT TTA GAA GTC CAT CGT TTG GTT CTG GGA GAA TTT AGA AAT | |
| ACA TAA GTT CCA GAC CGA CCA AAT CTT CAG GTA GCA AAC CAA GAC CCT CTT AAA TCT TTA | |
| Cys Ile Gln Gly Leu Ala Gly Leu Glu Val His Arg Leu Val Leu Gly Glu Phe Arg Asn | |
| 261 LRR8 266 267 LRR9 280 | |
| GAA GGA AAC TTG GAA AAG TTT GAC AAA TCT GCT CTA GAG GGC CTG TGC AAT TTG ACC ATT | |
| CTT CCT TTG AAC CTT TTC AAA CTG TTT AGA CGA GAT CTC CCG GAC ACG TTA AAC TGG TAA | |
| Glu Gly Asn Leu Glu Lys Phe Asp Lys Ser Ala Leu Glu Gly Leu Cys Asn Leu Thr Ile | |
| 281 285 LRR9 296 297 LRR10 300 | |
| GAA GAA TTC CGA TTA GCA TAC TTA GAC TAC TAC CTC GAT GAT ATT ATT GAC TTA TTT AAT | |
| CTT CTT AAG GCT AAT CGT ATG AAT CTG ATG ATG GAG CTA CTA TAA TAA CTG AAT AAA TTA | |
| Glu Glu Phe Arg Leu Ala Tyr Leu Asp Tyr Tyr Leu Asp Asp Ile Ile Asp Leu Phe Asn | |
| 301 305 LRR10 315 320 | |
| TGT TTG ACA AAT GTT TCT TCA TTT TCC CTG GTG AGT GTG ACT ATT GAA AGG GTA AAA GAC | |
| ACA AAC TGT TTA CAA AGA AGT AAA AGG GAC CAC TCA CAC TGA TAA CTT TCC CAT TTT CTG | |
| Cys Leu Thr Asn Val Ser Ser Phe Ser Leu Val Ser Val Thr Ile Glu Arg Val Lys Asp | |
| 321 325 LRR11 335 340 | |
| TTT TCT TAT AAT TTC GGA TGG CAA CAT TTA GAA TTA GTT AAC TGT AAA TTT GGA CAG TTT | |
| AAA AGA ATA TTA AAG CCT ACC GTT GTA AAT CTT AAT CAA TTG ACA TTT AAA CCT GTC AAA | |
| Phe Ser Tyr Asn Phe Gly Trp Gln His Leu Glu Leu Val Asn Cys Lys Phe Gly Gln Phe | |
| 341 343 344 LRR12 355 360 | |
| CCC ACA TTG AAA CTC AAA TCT CTC AAA AGG CTT ACT TTC ACT TCC AAC AAA GGT GGG AAT | |
| GGG TGT AAC TTT GAG TTT AGA GAG TTT TCC GAA TGA AAG TGA AGG TTG TTT CCA CCC TTA | |
| Pro Thr Leu Lys Leu Lys Ser Leu Lys Arg Leu Thr Phe Thr Ser Asn Lys Gly Gly Asn | |
| 361 363 364 LRR13 375 380 | |
| GCT TTT TCA GAA GTT GAT CTA CCA AGC CTT GAG TTT CTA GAT CTC AGT AGA AAT GGC TTG | |
| CGA AAA AGT CTT CAA CTA GAT GGT TCG GAA CTC AAA GAT CTA GAG TCA TCT TTA CCG AAC | |
| Ala Phe Ser Glu Val Asp Leu Pro Ser Leu Glu Phe Leu Asp Leu Ser Arg Asn Gly Leu | |
| 381 LRR13 385 386 LRR14 395 400 | |
| AGT TTC AAA GGT TGC TGT TCT CAA AGT GAT TTT GGG ACA ACC AGC CTA AAG TAT TTA GAT | |
| TCA AAG TTT CCA ACC ACA AGA GTT TCA CTA AAA CCC TGT TGG TCG GAT TTC ATA AAT CTA | |
| Ser Phe Lys Gly Cys Cys Ser Gln Ser Asp Phe Gly Thr Thr Ser Leu Lys Tyr Leu Asp | |
| 401 LRR14 411 412 LRR15 420 | |
| CTG AGC TTC AAT GGT GTT ATT ACC ATG AGT TCA AAC TTC TTG GGC TTA GAA CAA CTA GAA | |
| GAC TCG AAG TTA CCA CAA TAA TGG TAC TCA AGT TTG AAG AAC CCG AAT CTT GTT GAT CTT | |
| Leu Ser Phe Asn Gly Val Ile Thr Met Ser Ser Asn Phe Leu Gly Leu Glu Gln Leu Glu | |
| 421 LRR15 434 435 LRR16 440 | |
| CAT CTG GAT TTC CAG CAT TCC AAT TTG AAA CAA ATG AGT GAG TTT TCA GTA TTC GTA TCA | |
| GTA GAC CTA AAG GTC GTA AGG TTA AAC TTT GTT TAC TCA CTC AAA AGT CAT AAG CAT AGT | |
| His Leu Asp Phe Gln His Ser Asn Leu Lys Gln Met Ser Glu Phe Ser Val Phe Leu Ser | |
| 441 LRR16 459 460 | |
| CTC AGA AAC CTC ATT TAC CTT GAC ATT TCT CAT ACT CAC ACC AGA GTT GCT TTC AAT GGC | |
| GAG TCT TTG GAG TAA ATG GAA CTG TAA AGA GTA TGA GTG TGG TCT CAA CGA AAG TTA CCG | |
| Leu Arg Asn Leu Ile Tyr Leu Asp Ile Ser His Thr His Thr Arg Val Ala Phe Asn Gly | |
| 461 465 LRR17 475 480 | |
| ATC TTC AAT GGC TTG TCC AGT CTC GAA GTC TTG AAA ATG GCT GGC AAT TCT TTC CAG GAA | |
| TAG AAG TTA CCG AAC AGG TCA GAG CTT CAG AAC TTT TAC CGA CCG TTA AGA AAG GTC CTT | |
| Ile Phe Asn Gly Leu Ser Ser Leu Glu Val Leu Lys Met Ala Gly Asn Ser Phe Gln Glu | |
| 481 483 484 LRR18 495 500 | |
| AAC TTC CTT CCA GAT ATC TTC ACA GAG CTG AGA AAC TTG ACC TTC CTG GAC CTC TCT CAG | |
| TTG AAG GAA GGT CTA TAG AAG TGT CTC GAC TCT TTG AAC TGG AAG GAC CTG GAG AGA GTC | |
| Asn Phe Leu Pro Asp Ile Phe Thr Glu Leu Arg Asn Leu Thr Phe Leu Asp Leu Ser Gln | |
| 501 LRR18 508 509 LRR19 520 | |
| TGT CAA CTG GAG CAG TTG TCT CCA ACA GCA TTT AAC TCA CTC TCC AGT CTT CAG GTA CTA | |
| ACA GTT GAC CTC GTC AAC AGA GGT TGT CGT AAA TTG AGT GAG AGG TCA GAA GTC CAT GAT | |
| Cys Gln Leu Glu Gln Leu Ser Pro Thr Ala Phe Asn Ser Leu Ser Ser Leu Gln Val Leu | |
| 521 LRR19 532 533 LRR20 540 | |
| AAT ATG AGC CAC AAC AAC TTC TTT TCA TTG GAT ACG TTT CCT TAT AAG TGT CTG AAC TCC | |
| TTA TAC TCG GTG TTG TTG AAG AAA AGT AAC CTA TGC AAA GGA ATA TTC ACA GAC TTG AGG | |
| Asn Met Ser His Asn Asn Phe Phe Ser Leu Asp Thr Phe Pro Tyr Lys Cys Leu Asn Ser | |
| 541 LRR20 550 556 557 560 | |
| CTC AAA GAG CTC GCC CTG GAC ACC AAC CAG CTG AAG TCT GTT CCT GAT GGG ATT TTT GAT | |
| GAG TTT CTC GAG CGG GAC CTG TGG TTG GTC GAC TTC AGA CAA GGA CTA CCC TAA AAA CTA | |
| Leu Lys Glu Leu Ala Leu Asp Thr Asn Gln Leu Lys Ser Val Pro Asp Gly Ile Phe Asp | |
| 561 VLRB.6_hybrid1 580 | |
| CGC CTG ACC AGC TTG CAG AAA ATT TGG CTT CAT ACA AAC CCT TGG GAC TGC AGT TGT CCC | |
| GCG GAC TGG TCG AAC GTC TTT TAA ACC GAA GTA TGT TTG GGA ACC CTG ACG TCA ACA GGG | |
| Arg Leu Thr Ser Leu Gln Lys Ile Trp Leu His Thr Asn Pro Trp Asp Cys Ser Cys Pro | |
| 581 VLRB.61_hybrid1 600 | |
| AGG ATT GAC TAT CTC AGT CGC TGG TTA AAT AAA AAC TCA CAG AAG GAA CAG GGC AGT GCT | |
| TCC TAA CTG ATA GAG TCA GCG ACC AAT TTA TTT TTG AGT GTC TTC CTT GTC CCG TCA CGA | |
| Arg Ile Asp Tyr Leu Ser Arg Trp Leu Asn Lys Asn Ser Gln Lys Glu Gln Gly Ser Ala | |
| 601 VLRB.61_hybrid1 620 | |
| AAA TGT TCC GGG TCC GGC AAG CCC GTC CGA AGT ATC ATC TGC CCT ACT CTC GAG GAC AAA | |
| TTT ACA AGG CCC AGG CCG TTC GGG CAG GCT TCA TAG TAG ACG GGA TGA GAG CTC CTG TTT | |
| Lys Cys Ser Gly Ser Gly Lys Pro Val Arg Ser Ile Ile Cys Pro Thr Leu Glu Asp Lys | |
| 621 VLRB.61_hybrid1 636 637 hFC DOMAIN_{—} | |
| ACT CAC ACA TGC CCA CCG TGC CCA GCA CCT GAA CTC CTG GGG GGA CCG TCA GTC TTC CTC | |
| TGA GTG TGT ACG GGT GGC ACG GGT CGT GGA CTT GAG GAC CCC CCT GGC AGT CAG AAG GAG | |
| Thr His Thr Cys Pro Pro Cys Pro Ala Pro Glu Leu Leu Gly Gly Pro Ser Val Phe Leu | |
| 641 645 hFC DOMAIN 655 660 | |
| TTC CCC CCA AAA CCC AAG GAC ACC CTC ATG ATC TCC CGG ACC CCT GAG GTC ACA TGC GTG | |
| AAG GGG GGT TTT GGG TTC CTG TGG GAG TAC TAG AGG GCC TGG GGA CTC CAG TGT ACG CAC | |
| Phe Pro Pro Lys Pro Lys Asp Thr Leu Met Ile Ser Arg Thr Pro Glu Val Thr Cys Val | |
| 661 665 hFC DOMAIN 675 680 | |
| GTG GTG GAC GTG AGC CAC GAA GAC CCT GAG GTC AAG TTC AAC TGG TAC GTG GAC GGC GTG | |
| CAC CAC CTG CAC TCG GTG CTT CTG GGA CTC CAG TTC AAG TTG ACC ATG CAC CTG CCG CAC | |
| Val Val Asp Val Ser His Glu Asp Pro Glu Val Lys Phe Asn Trp Tyr Val Asp Gly Val | |
| 681 685 hFC DOMAIN 695 700 | |
| GAG GTG CAT AAT GCC AAG ACA AAG CCG CGG GAG GAG CAG TAC AAC AGC ACG TAC CGT GTG | |
| CTC CAC GTA TTA CGG TTC TGT TTC GGC GCC CTC CTC GTC ATG TTG TCG TGC ATG GCA CAC | |
| Glu Val His Asn Ala Lys Thr Lys Pro Arg Glu Glu Gln Tyr Asn Ser Thr Tyr Arg Val | |
| 701 705 hFC DOMAIN 715 720 | |
| GTC AGC GTC CTC ACC GTC CTG CAC CAG GAC TGG CTG AAT GGC AAG GAG TAC AAG TGC AAG | |
| CAG TCG CAG GAG TGG CAG GAC GTG GTC CTG ACC GAC TTA CCG TTC CTC ATG TTC ACG TTC | |
| Val Ser Val Leu Thr Val Leu His Gln Asp Trp Leu Asn Gly Lys Glu Tyr Lys Cys Lys | |
| 721 725 hFC DOMAIN 735 740 | |
| GTC TCC AAC AAA GCC CTC CCA GCC CCC ATC GAG AAA ACC ATC TCC AAA GCC AAA GGG CAG | |
| CAG AGG TTG TTT CGG GAG GGT CGG GGG TAG CTC TTT TGG TAG AGG TTT CGG TTT CCC GTC | |
| Val Ser Asn Lys Ala Leu Pro Ala Pro Ile Glu Lys Thr Ile Ser Lys Ala Lys Gly Gln | |
| 741 745 hFC DOMAIN 755 760 | |
| CCC CGA GAA CCA CAG GTG TAC ACC CTG CCC CCA TCC CGG GAG GAG ATG ACC AAG AAC CAG | |
| GGG GCT CTT GGT GTC CAC ATG TGG GAC GGG GGT AGG GCC CTC CTC TAC TGG TTC TTG GTC | |
| Pro Arg Glu Pro Gln Val Tyr Thr Leu Pro Pro Ser Arg Glu Glu Met Thr Lys Asn Gln | |
| 761 765 hFC DOMAIN 775 780 | |
| GTC AGC CTG AGC TGC CTG GTC AAA GGC TTC TAT CCC AGC GAC ATC GCC GTG GAG TGG GAG | |
| CAG TCG GAC TGG ACG GAC CAG TTT CCG AAG ATA GGG TCG CTG TAG CGG CAC CTC ACC CTC | |
| Val Ser Leu Thr Cys Leu Val Lys Gly Phe Tyr Pro Ser Asp Ile Ala Val Glu Trp Glu | |
| 781 785 hFC DOMAIN 795 800 | |
| AGC AAT GGG CAG CCG GAG AAC AAC TAC AAG ACC ACG CCT CCC GTG CTG GAC TCC GAC GGC | |
| TCG TTA CCC GTC GGC CTC TTG TTG ATG TTC TGG TGC GGA GGG CAC GAC CTG AGG CTG CCG | |
| Ser Asn Gly Gln Pro Glu Asn Asn Tyr Lys Thr Thr Pro Pro Val Leu Asp Ser Asp Gly | |
| 801 805 hFC DOMAIN 815 820 | |
| TCC TTC TTC CTC TAC AGC AAG CTC ACC GTG GAC AAG AGC AGG TGG CAG CAG GGG AAC GTC | |
| AGG AAG AAG GAG ATG TCG TTC GAG TGG CAC CTG TTC TCG TCC ACC GTC GTC CCC TTG CAG | |
| Ser Phe Phe Leu Tyr Ser Lys Leu Thr Val Asp Lys Ser Arg Trp Gln Gln Gly Asn Val | |
| 821 825 hFC DOMAIN 835 840 | |
| TTC TCA TGC TCC GTG ATG CAT GAG GCT CTG CAC AAC CAC TAC ACG CAG AAG AGC CTC TCC | |
| AAG AGT ACG AGG CAC TAC GTA CTC CGA GAC GTG TTG GTG ATG TGC GTC TTC TCG GAG AGG | |
| Phe Ser Cys Ser Val Met His Glu Ala Leu His Asn His Tyr Thr Gln Lys Ser Leu Ser | |
| 841 845 hFC DOMAIN 855 860 | |
| CTG TCT CCG GGT AAA TGA | |
| GAC AGA GGC CCA TTT ACT | |
| Leu Ser Pro Gly Lys *** | |
| 861 865 866 | |

What is claimed is:

1. An isolated nucleic acid molecule encoding a fusion polypeptide capable of binding myeloid differentiation protein-2 (MD-2) polypeptide comprising: a nucleotide sequence encoding an MD-2 polypeptide-binding portion of human toll-like receptor 4 (TLR4), a leucine-rich repeats (LRR) module, and a multimerizing component.

2. The nucleic acid molecule of claim 1, wherein the MD-2 polypeptide-binding portion of human TLR4 is ectodomain of human TLR4.

3. The nucleic acid molecule of claim 1, wherein the LRR module is obtained from variable lymphocyte receptors (VLR).

4. The nucleic acid molecule of claim 3, wherein the VLR is VLR-B.61.

5. The isolated nucleic acid molecule of claim 1, wherein the nucleotide sequence encoding MD-2 polypeptide-binding portion of human TLR4 is positioned upstream of the nucleotide sequence encoding the LRR module.

6. The isolated nucleic acid molecule of claim 1, wherein the multimerizing component comprises an immunoglobulin domain.

7. The isolated nucleic acid molecule of claim 6, wherein the immunoglobulin domain is selected from the group consisting of the Fc domain of IgG, the heavy chain of IgG, and the light chain of IgG.

8. The isolated nucleic acid molecule of claim 1, wherein the nucleic acid sequence encoding the MD-2 polypeptide-binding portion of TLR4 consists of a nucleotide sequence selected from the group consisting of:
(a) the nucleotide sequence of from 115 to 2598 of SEQ ID NO:1 set forth in Table 1 referred to as TFL;
(b) the nucleotide sequence of from 115 to 1641 of SEQ ID NO:3 set forth in Table 1 referred to as TOY3;
(c) the nucleotide sequence of from 115 to 2523 of SEQ ID NO:5 set forth in Table 1 referred to as TOY8;
(d) the nucleotide sequence of from 115 to 2595 of SEQ ID NO:7 set forth in Table 1 referred to as TOY9; and
(e) a nucleotide sequence which, as a result of the degeneracy of the genetic code, differs from the nucleotide sequence of (a), (b), (c), or (d).

9. An expression vector which comprises the nucleic acid molecule of claim 1.

10. A host-vector system for the production of a fusion polypeptide which comprises the expression vector of claim 9, in a suitable host cell.

11. The host-vector system of claim 10, wherein the suitable host cell is a bacterial cell, yeast cell, insect cell, or mammalian cell.

12. A method of producing a fusion polypeptide which comprises growing cells of the host-vector system of claim 10, under conditions permitting production of the fusion polypeptide and recovering the fusion polypeptide so produced.

13. A fusion polypeptide encoded by the isolated nucleic acid molecule of claim 1.

14. A composition comprising a molecule capable of binding MD-2 molecule to form a nonfunctional complex comprising a multimer of the fusion polypeptide of claim 13.

15. The composition of claim 14, wherein the multimer is a dimer.

16. The fusion polypeptide of claim 13, which has been modified by acetylation or pegylation.

17. A method of decreasing or inhibiting inflammatory response in a mammal comprising administering an effective amount of the fusion polypeptide of claim 13 to the mammal in need thereof.

18. A method of attenuating or preventing sepsis or septic shock in a mammal comprising administering an effective amount of the fusion polypeptide of claim 13 to the mammal in need thereof.

19. The method of claim 18, wherein the sepsis or septic shock is characterized by vasodilation and extravascular plasma leakage resulting from an increase in endothelial permeability.

20. A method of inhibiting TLR4 ligand activities in a mammal comprising administering an effective amount of the fusion polypeptide of claim 13 to the mammal in need thereof.

21. The method of claim 20, wherein TLR4 ligand activity causes penetrating trauma to the abdomen.

22. The method of claim 20, wherein TLR4 ligand activity causes large bowel incarceration.

23. The method of claim 20, wherein TLR4 ligand activity causes rheumatoid arthritis.

24. The nucleic acid molecule of claim 1, wherein the MD-2 polypeptide-binding portion of human TLR4 is A patch of human TLR4.
